(19) United States
(12) Patent Application Publication
(10) Pub. No.: US 2026/0334806 A1
(43) Pub. Date: Oct. 29, 2026

(54) MICROMECHANICAL RESONATORS

(71) Applicant: Northern Digital Inc., Waterloo (CA)

(72) Inventors: Gereon Vogtmeier, Aachen (DE); Bernhard Gleich, Hamburg (DE); Peter Cäsar Mazurkewitz, Hamburg (DE); Pouyan Sadeghian, Hamburg (DE); Christian Jörg Schilling, Thayngen (CH)

(21) Appl. No.: 19/276,522

(22) Filed: Jul. 22, 2025

Related U.S. Application Data
(60) Provisional application No. 63/793,271, filed on Apr. 23, 2025.
(60) Provisional application No. 63/793,237, filed on Apr. 23, 2025.

(51) Int. Cl. A61B 5/06; A61B 34/20

(52) U.S. Cl. CPC A61B 5/062; A61B 2034/2072

(57) ABSTRACT

Micromechanical resonators can include an elongated housing that includes a groove at a longitudinal center of a first end the housing. A first end of a filament can be disposed within the groove and attached to the elongated housing. A first permanent magnet can be disposed in the housing and connected to a second end of the filament. The first permanent magnet can include a cylindrical portion. A second permanent magnet can be disposed in the housing and attached to the housing at a second end of the housing. The second permanent magnet can include a cylindrical portion and be axially spaced apart from the first permanent magnet. The filament can be configured to allow the first permanent magnet to oscillate about an equilibrium position with respect to the second permanent magnet in response to a magnetic or electromagnetic field.

### CLAIM OF PRIORITY

This application claims priority under 35 USC § 119(e) to U.S. patent application Ser. No. 63/793,237, filed on Apr. 23, 2025, and U.S. patent application Ser. No. 63/793,271, filed on Apr. 23, 2025, the entire contents of which are hereby incorporated by reference.

### TECHNICAL FIELD

The present disclosure relates to passive micromechanical resonators that can produce a detectable signal in a magnetic or electromagnetic field that can be sensed externally of the micromechanical resonator to determine a position and/or orientation of micromechanical resonator.

### BACKGROUND

It is often desirable to track markers within an anatomy of a patient to diagnose medical conditions. Some techniques include imaging (e.g., an angiogram, ultrasound, tomography, etc.) to track the markers. Other techniques use electromagnetic sensors to track the markers. For example, an electromagnetic sensor can detect an electric or magnetic field generated by a marker and determine the position of the marker based on the detected electric or magnetic field.

### SUMMARY

Micromechanical resonators (MMR) also referred to as “MicroBot” devices are small magnetic resonator devices that can have a typical size of less than 10 mm in length and a diameter of 0.4-1.5 mm. However, sizes and diameters larger and smaller than these ranges can be used. Inside a housing, two or three magnetic objects (e.g., spherical permanent magnets, cylindrical permanent magnets, coils, etc.) are mounted in a way that one magnetic object can mechanically oscillate in a magnetic or electromagnetic field with a defined distance from the other magnetic object(s) due to an excitation of a resonance mode. The medical oscillation of the magnetic object can be detected externally of the micromechanical resonator by a sensor to determine the position and/or location of the of the micromechanical resonator. The micromechanical resonators receive energy from an external magnetic or electromagnetic field and do not need a wired connection to an energy supply. Micromechanical resonators are completely passive devices and do not require electronics.

The micromechanical resonators described herein include at least one free (e.g., non-fixed) magnetic object arranged in mechanical equilibrium with a second magnetic object (e.g., a second free magnetic object, a fixed magnetic object, etc.). In the presence of an external magnetic or electromagnetic field (e.g., generated by a transducer in a transmit mode), the mechanical equilibrium of the magnetic objects is disturbed, and the free magnetic object oscillates in a rotational mode relative to the second magnetic objects(s) at one or more resonant frequencies to produce a detectable signal (e.g., a pulse) within the magnetic or electromagnetic field. After turning off the magnetic or electromagnetic field, the free magnetic object continues oscillating in the rotational mode and continues emitting the detectable signal in the magnetic or electromagnetic field. The detectable signal can be sensed by a receiver (e.g., the transducer operating in a receive mode) and a computer system can determine one or more properties (e.g., a position, orientation, spatial (e.g., 2D or 3D) coordinates, etc.) of the micromechanical resonator based on detectable signals from the micromechanical resonator. Details of micromechanical resonators are described in U.S. Pat. Nos. 11,598,677 and 11,774,300, and U.S. Publication No. 2025/0127417A1 which are hereby incorporated by reference in their entirety. Additional micromechanical resonators are described in U.S. Application No. filed contemporaneously with this application which is also hereby incorporated by reference in its entirety.

Low friction between the oscillating magnetic object and objects (magnetic and non-magnetic) in contact with the oscillating magnetic object is important. Low friction helps to provide a long and measurable oscillation after turning off the external magnetic or electromagnetic field. For example, the oscillating magnetic object can be held in position by a thin filament which twists during oscillation of the oscillating magnetic object. Having a filament that is resilient (e.g., not brittle) in torsion (e.g., by a low torsional stiffness) and stiff under uniaxially loading can be helpful.

This disclosure describes a method for manufacturing a housing for the micromechanical resonator and fixtures (e.g., a carrier for magnetic objects inside the housing, an attachment device for attaching a filament to a magnetic object, etc.) using 3D printing technologies. The housing can include an opening for filament feedthrough (e.g., under the force of a vacuum pump). The housing can include one or more resilient arms for releasably holding a magnetic object within the housing during assembly and tuning of a resonance frequency of the micromechanical resonator.

The dimensions and the precision for the fabrication are challenging and conventional manufacturing methods can be limiting. 3D printing with a 2-photon polymerization technology allows precise printing of features with a resolution down to 200 nm. 3D printing with a 2-photon polymerization technology in combination with a printing compatible design and geometry that allows for assembly of a two (or three) magnet resonator as described herein.

In a first aspect, a device (e.g., a micromechanical resonator) includes an elongated housing including a groove at a longitudinal center of a first end the elongated housing. The device includes filament having a first end disposed within the groove and being attached to the elongated housing. A first permanent magnet is disposed in the elongated housing and is connected to a second end of filament. The first permanent magnet has a cylindrical portion. A second permanent magnet is disposed in the elongated housing and is attached to the elongated housing at a second end of the elongated housing. The second permanent magnet has a cylindrical portion and is axially spaced apart from the first permanent magnet. The filament is configured to allow the first permanent magnet to oscillate about an equilibrium position with respect to the second permanent magnet in response to a magnetic or electromagnetic field.

In some implementations, a lateral side of the groove is open to an enlarged opening of the elongated housing.

In some implementations, the enlarged opening is configured to allow axial insertion of the filament through the first end of the elongated housing before the filament is laterally moved into the groove.

In some implementations, the groove is recessed into a sidewall of elongated housing defining the enlarged opening.

In some implementations, the enlarged opening is disposed within a reservoir at the first end of the elongated housing.

In some implementations, the elongated housing includes one or more resilient arms disposed at the second end of the elongated housing. The one or more resilient arms are configured to apply a radially inward force to the second permanent magnet for holding the second permanent magnet in a fixed location within the elongated housing.

In some implementations, the one or more resilient arms extend in a longitudinal direction and surround the second permanent magnet.

In some implementations, an axial end of the one or more resilient arms has a tapered internal diameter to allow outward radial deflection of the one or more resilient arms when the second permanent magnet is inserted into the elongated housing, and the outward radial deflection of the one or more resilient arms causes the radially inward force on the second permanent magnet to be applied.

In some implementations, the device is configured such that prior to the second permanent magnet being adhered to the one or more resilient arms, the one or more resilient arms (i) allow the second permanent magnet to move axially relative to the elongated housing when an axial force exerted on the second permanent magnet exceeds a threshold force and (ii) hold the second permanent magnet axially relative to the elongated housing when the axial force exerted on the second permanent magnet is below the threshold force.

In some implementations, the device is configured such that prior to the second permanent magnet being adhered to the one or more resilient arms, an axial movement of the second permanent magnet relative to the elongated housing changes a resonant frequency in which the first permanent magnet oscillates about the longitudinal axis of the elongated housing relative to the second permanent magnet.

In some implementations, the device includes a first part attached to (i) a first end of the first permanent magnet and (ii) the second end of the filament.

In some implementations, the first part is configured to concentrically align the first permanent magnet to the elongated housing.

In some implementations, the first part includes one or more openings extending through the first part, and the filament extends through the one or more openings of the first part.

In some implementations, at least one opening of the one or more openings has a narrow portion located at a longitudinal axis of the first part and a wide portion that is open to the narrow portion. The narrow portion is open at an outer perimeter of the first part for allowing lateral insertion of the filament into the first part.

In some implementations, the at least one opening is tapered along the longitudinal axis of the first part.

In some implementations, the one or more openings of the first part are configured to allow the filament to be inserted into the one or more openings in a lateral direction that is perpendicular to the longitudinal axis of the first part.

In some implementations, the one or more openings of the first part include two or more openings that are spaced apart from one another around the longitudinal axis of the first part.

In some implementations, the filament extends through the two or more openings and is at least partially wound around the first part.

In some implementations, the first part includes a first axially-extending protrusion, and the filament is attached to the first axially-extending protrusion.

In some implementations, the first part includes a plurality of distance elements integrally formed with the first part. The plurality of distance elements are configured to allow the first part to be substantially parallel to the first permanent magnet when the first part is in contact with the first permanent magnet.

In some implementations, a magnetic axis of the first permanent magnet is oriented in a first direction that is perpendicular to the longitudinal axis of the elongated housing, and a magnetic axis of the second permanent magnet is oriented in a second direction that is opposite to the first direction.

In some implementations, the first direction is perpendicular to a longitudinal axis of the cylindrical portion of the first permanent magnet, and the second direction is perpendicular to a longitudinal axis of the cylindrical portion of the second permanent magnet.

In some implementations, the filament includes a Kevlar® fiber, an ultra-high density polyethylene fiber, a carbon fiber, or an aramid fiber.

In some implementations, a torsional stiffness of the filament is less than 1e-7 Nm/rad, and an axial stiffness of the filament is greater than 1 GPa.

In some implementations, at least one of the first permanent magnet or the second permanent magnet has a tapered cylindrical portion.

In some implementations, the first permanent magnet has a first tapered cylindrical portion, the second permanent magnet has a second tapered cylindrical portion, and the first and second tapered cylindrical portions directly face one another.

In a second aspect, a device includes an elongated housing including a groove at a longitudinal center of a first end the elongated housing. A lateral side of the groove is open to an enlarged opening of the elongated housing. The device includes a filament having a first end disposed within the groove and being attached to the elongated housing. A first magnetic object is disposed in the elongated housing and is connected to a second end of filament. A second magnetic object is disposed in the elongated housing and is connected to the elongated housing at a second end of the elongated housing with the second magnetic object being axially spaced apart from the first magnetic object. The filament is configured to allow the first magnetic object to oscillate about an equilibrium position with respect to the second magnetic object in response to a magnetic or electromagnetic field.

In some implementations, the second magnetic object is a spherical permanent magnet with a magnetic axis oriented in a first direction that is perpendicular to the longitudinal axis of the elongated housing.

In some implementations, the first magnetic object is a spherical permanent magnet with a magnetic axis oriented in a second direction that is perpendicular to the longitudinal axis of the elongated housing and opposite to the first direction.

In some implementations, the device includes a second part disposed within the housing with the second magnetic object disposed in the second part.

In some implementations, the elongated housing is cylindrical and at least one of the first end of the elongated housing or the second end of the elongated housing is tapered.

In some implementations, the first end of the elongated housing is tapered relative to a cylindrical portion of the elongated housing, and the second end of the elongated housing is tapered relative to the cylindrical portion of the elongated housing.

In some implementations, the second magnetic object is a cylindrical permanent magnet with a magnetic axis oriented in a first direction that is perpendicular to the longitudinal axis of the housing.

In some implementations, the first magnetic object is a cylindrical permanent magnet with a magnetic axis oriented in a second direction that is perpendicular to the longitudinal axis of the housing and opposite to the first direction.

In some implementations, the second magnetic object has a longitudinal length of less than 2 mm, and the first magnetic object has a longitudinal length greater than 0.5 mm.

In some implementations, a longitudinal length of the elongated housing is less than 10 mm.

In some implementations, the device includes a first part adhered to the first magnetic object and to the second end of the filament.

In some implementations, the first part is adhered to the first magnetic object by a cyanoacrylate, an epoxy-based adhesive, or a UV curable adhesive, and the first part is adhered to the second end of the filament by the cyanoacrylate, the epoxy-based adhesive, or the UV curable adhesive.

In some implementations, the enlarged opening has a tapered sidewall.

In some implementations, the filament includes a Kevlar® fiber, an ultra-high density polyethylene fiber, a carbon fiber, or an aramid fiber.

In some implementations, the elongated housing is a monolithic structure.

In some implementations, at least one of the first or second magnetic objects includes a coil.

In a third aspect, a system includes one or more devices. Each device includes a housing and a filament disposed in the housing with a first end of the filament attached to the housing. Each device includes a first permanent magnet disposed in the housing and being connected to a second end of the filament. The first permanent magnet has a cylindrical portion. Each device includes a second permanent magnet disposed in the housing and being attached to the housing at a second end of the housing. The second permanent magnet has a cylindrical portion and is axially spaced apart from the first permanent magnet. The filament is configured to allow the first permanent magnet to oscillate about an equilibrium position with respect to the second permanent magnet in response to a magnetic or electromagnetic field. Each device has a different resonant frequency in which the first permanent magnet oscillates about the equilibrium position with respect to the second permanent magnet.

In some implementations, each device has a different resonant frequency due to different spacing between the respective first and second permanent magnets.

In some implementations, the system includes one or more electromagnetic sources external to the one or more devices. The one or more electromagnetic sources configured to provide respective magnetic or electromagnetic fields to excite the respective resonant frequencies.

In some implementations, the system includes one or more sensors external to the one or more devices, the one or more sensors being configured to determine respective positions of the one or more devices based on sensing the respective resonant frequencies.

In some implementations, the system includes a medical device. The one or more devices are disposed in the medical device, and the one or more sensors are configured to determine a position of the medical device based on sensing the respective resonant frequencies.

In some implementations, the medical device is a flexible catheter, and the one or more devices are disposed in a sidewall of the flexible catheter.

In some implementations, for each device, an opening in the housing is configured to allow the filament to be inserted into the housing prior to the first end of the filament being attached and fixed to the first end of the housing. The opening is located at a longitudinal end of the housing, the opening including a groove and an enlarged portion.

In some implementations, for each device, a magnetic axis of the first permanent magnet is perpendicular to a longitudinal axis of the housing, a magnetic axis of the second permanent magnet is perpendicular to the longitudinal axis of the housing, and the magnetic axis of the first permanent magnet and the magnetic axis of the second permanent magnet have opposite polarities.

In some implementations, the cylindrical portion of the first permanent magnet and the cylindrical portion of the second permanent magnet have respective longitudinal axes oriented parallel to the longitudinal axis of the housing.

In a fourth aspect, a method includes inserting a filament through a first opening of an elongated housing in a longitudinal direction. The first opening disposed at a first end of the elongated housing. With the filament inserted in the first opening, the filament is laterally moved into a groove of the elongated housing. The groove located at a longitudinal center of the elongated housing and being open to the first opening. With the filament inserted in the groove, the filament is adhered to the elongated housing. An end of the filament is connected to a first permanent magnet. The first permanent magnet has a cylindrical portion. The first permanent magnet is inserted into the elongated housing. A second permanent magnet is inserted into the elongated housing to locate the second permanent magnet in a position that is spaced apart from the first magnet. The second permanent magnet has a cylindrical portion.

In some implementations, the method includes tuning a resonant frequency in which the first permanent magnet oscillates relative to the second permanent magnet in response to a magnetic or electromagnetic field.

In some implementations, tuning the resonant frequency includes varying an axial position of the second permanent magnet within the housing while one or more resilient arms of the elongated housing apply a radially inward force to the second permanent magnet.

In some implementations, after tuning the resonant frequency, the method includes adhering the one or more resilient arms to the second permanent magnet to fix the axial position of the second permanent magnet and the resonant frequency.

In some implementations, the method includes tuning the resonant frequency by varying an axial position of the second permanent magnet within the housing while the filament is adhered to the groove.

In some implementations, the elongated housing has been 3D printed by 2-photon polymerization technology.

In some implementations, the method includes adhering, by a cyanoacrylate, an epoxy-based adhesive, or a UV curable adhesive, the filament within the groove.

In a fifth aspect, a method includes printing, using a 3D printing technology, an elongated housing including (i) a first opening located at a first end of the elongated housing, and (ii) one or more axially-extending resilient arms located at a second end of the housing. The first opening includes a groove that is recessed relative to an enlarged opening and open to the enlarged opening. The groove is located at a longitudinal center of the elongated housing.

In some implementations, a longitudinal length of the elongated housing is less than 10 mm.

In some implementations, a longitudinal length of the housing is between 2 and 5 mm.

In some implementations, the 3D printing technology is a 2-photon polymerization technology.

In some implementations, the first end of the elongated housing is tapered relative to a cylindrical portion of the elongated housing, and the second end of the elongated housing is tapered relative to the cylindrical portion of the elongated housing.

In some implementations, the enlarged opening has a tapered wall.

In some implementations, the method includes printing, using the 3D printing technology, a first part configured to be disposed within the elongated housing. The first part having a through opening at a longitudinal center of the first part.

In some implementations, at least one wall defining the through opening is tapered toward a longitudinal axis of the first part.

While specific implementations have been described for specific aspects, any of the above-noted implementations and features of the above aspects can be combined with any of the other aspects. For example, features and/or implementations described with reference to the first aspect can be used with second aspect, and vice versa, etc. In addition, any combination of the above-noted aspects is also within the scope of this disclosure.

The technology described herein can include one or more of the following advantages.

A housing having a first end that includes a groove located at a longitudinal center of the housing and an enlarged opening that is open to the groove can improve manufacturability of the mechanical resonators described herein. For example, a filament can be axially inserted through the enlarged opening, then laterally moved into the groove for alignment, and then adhered (e.g., glued) within the groove to attach the filament to housing so the filament is located at the longitudinal center of the housing.

A housing having one or more resilient arms that apply a radially inward force to a second magnetic object within the housing can improve the practicality of tuning a resonant frequency of the micromechanical resonator. For example, the second magnetic object can be held in a first position by the one or more resilient arms and the resonant frequency in which a first magnetic object oscillates relative to the second magnetic object determined (e.g., by the processes described herein). If the resonant frequency is unsatisfactory, an axial force can be applied to the second magnetic object (e.g., by pushing) to overcome the sliding friction between the resilient arms and the second magnetic object to move the second magnetic object relative to the housing. For example, the second magnetic object can be moved closer to the first magnetic object within the housing to increase the resonant frequency and/or the second magnetic object can be moved further from the first magnetic object within the housing to decrease the resonant frequency. Once the resonant frequency is satisfactory, an adhesive can be applied between the resilient arms and the second magnetic object to fix the axial location of the second magnetic object within the housing.

Having the first magnetic object implemented as a first cylindrical permanent magnet and the second magnetic object implemented as a second cylindrical permanent magnet can provide a stronger signal relative to the size of the micromechanical resonator than the same size micromechanical resonator with spherical permanent magnets. This can be helpful to improve the detection of the micromechanical resonators, which are typically very small (e.g., on the order of millimeters).

Having a first (or second) magnetic object with a tapered portion that faces toward the other of the second (or first) magnetic object can provide better field guidance of the magnetic objects. In some cases, having both the first and second magnetic objects with respective tapered portions that directly face one another can provide better field guidance of the magnetic objects compared to magnetic objects without tapered portions.

Using a first part (e.g., an attachment part) to attach the filament to the first magnetic object can provide a strong connection between the filament and the first magnetic object. For example, a first part with one or more openings for the filament to extend through and be adhered to can be stronger than adhering an axial end of the filament to the first magnetic object directly. Similarly, a first part with a protrusion in which the filament can be adhered to can be stronger than adhering the filament to the first magnetic object directly. In addition, the first part can have a contour that substantially corresponds to a shape of the first magnetic object which can provide a large, distributed area for adhesion and alignment between the first part and the first magnetic object.

A first part with distance elements can help to ensure the first part is in parallel alignment with the first magnetic object while allowing axial clearance for the filament to traverse perpendicularly relative to the first part and between the first part and the first magnetic object.

Using a second part (e.g., a carrier) to hold the second magnetic object within the housing can be helpful in implementations in which the second magnetic object is a spherical permanent magnet while allowing the second magnetic object to axially move relative to the housing during frequency tuning.

Using a filament with a low torsional stiffness while being resilient to rotation can be helpful to limit the filament's influence on the resonant frequency in which a first magnetic object oscillates relative to a second magnetic object and allow flexibility in the assembly process. For example, if too stiff of a filament is used, the filament can break during assembly and would need to be replaced.

Printing the housing using a 2-photon polymerization technology can allow the housing to be printed with the groove, enlarged opening, and one or more resilient arms directly instead of requiring a secondary manufacturing method to produce them. These features may be on the order of microns wide, which can be difficult to manufacture otherwise.

Designing the housing with radial clearance between the free magnetic object and the sidewall of the housing is helpful to limit the likelihood of the first (or free) magnetic object hitting the sidewall of the housing while oscillating in the rotational resonance mode. While it has been found that gravitational influence on the free magnetic object reduces with the size of the micromechanical resonators, it is nevertheless helpful to provide clearance between the free magnetic object and the sidewall of the housing. For example, a free magnetic object in a large micromechanical resonator (e.g., on the order of 100 cm) may impact the sidewall of the housing during shaking of the micromechanical resonator. However, a free magnetic object in a small micromechanical resonator (e.g., on the order of 1 mm) is very unlikely to impact the sidewall of the housing during shaking of the micromechanical resonator due to the relative strength of the magnetic force compared to the gravitational force.

The details of one or more implementations are set forth in the accompanying drawings and the description herein. Other features, objects, and advantages will be apparent from the description and drawings, and from the claims.

### DESCRIPTION OF THE DRAWINGS

FIG. 1 shows a micromechanical resonator being used in a medical environment.

FIG. 2 is a schematic of a micromechanical resonator with two magnetic objects implemented as cylindrical permanent magnets.

FIG. 3 is a time-domain plot illustrating voltages of an external magnetic or electromagnetic field.

FIG. 4 is a frequency-domain plot illustrating resonances of a micromechanical resonator.

FIGS. 5 and 6 are views of a cylindrical housing of a micromechanical resonator.

FIG. 7 shows resilient arms of the cylindrical housing.

FIGS. 8 and 9 are views of a groove and an enlarged opening for filament feedthrough and center alignment of the filament.

FIG. 10 shows a first part (e.g., an attachment part) for supporting and aligning the first magnetic object within the cylindrical housing.

FIG. 11 shows a first part (e.g., an attachment part) having a tapered opening for the filament and a plurality of axially-extending protrusions.

FIG. 12 is a cross-section of the first part of FIG. 11 showing the tapered opening in further detail.

FIG. 13 is a view of a tapered first end of the cylindrical housing.

FIG. 14 is a view of a tapered second end of the cylindrical housing.

FIG. 15 is an example method for assembling a micromechanical resonator.

FIG. 16 shows a first part (e.g., an attachment part) with the filament wound around the first part.

FIG. 17 shows a first part (e.g., an attachment part) with the protrusion for filament attachment.

FIG. 18 shows a micromechanical resonator with two magnetic objects, each having a tapered portion that faces one another.

FIG. 19 shows a micromechanical resonator with one magnetic object having a tapered portion.

FIG. 20 shows a micromechanical resonator with the first magnetic object of FIG. 19 with a second magnetic object that includes a spherical portion disposed in a second part (e.g., a carrier).

FIG. 21 shows a micromechanical resonator with the second magnetic object of FIG. 20 with a first magnetic object that includes a spherical portion and a first part (e.g., an attachment part) contoured to match the shape of the spherical portion.

FIG. 22 shows a micromechanical resonator with two movable magnetic objects.

FIG. 23 shows an example computer device and example mobile computer device which can be used to implement the techniques described herein.

Like reference numbers represent corresponding parts throughout.

### DETAILED DESCRIPTION

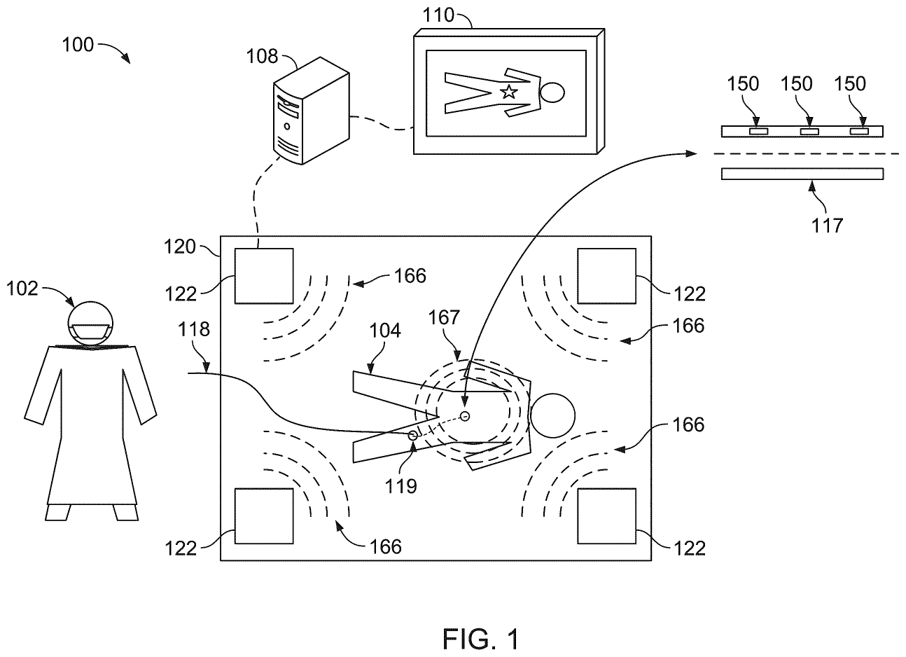

FIG. 1 shows a medical environment 100 where a medical professional 102 desires to know the position and/or orientation of a device 150 (e.g., a micromechanical resonator, marker device, sensor, etc.) within the anatomy of a patient 104 during a medical procedure (e.g., a minimally invasive procedure). For example, in the example represented in FIG. 1, the medical professional 102 has inserted a catheter 118 into the patient 104 via an insertion site 119. In this example, three micromechanical resonators 150 are embedded in a sidewall 117 of the catheter 118. In other examples, a single micromechanical resonator 150 can be used, or more generally one or more micromechanical resonators 150 can be used.

Encapsulating the micromechanical resonator 150 in the sidewall 117 of the catheter 118 can protect the magnets and inner components of the micromechanical resonator 150 from being exposed to bodily fluids and/or otherwise being contaminated. The catheter 118 is generally a thin-walled flexible tube formed of non-magnetic bio-compatible material (polyethylene, silicone). The micromechanical resonator 150 can be very small (e.g., 1 mm in wall thickness or less) so it can fit inside the catheter 118 and allow the functionality of the catheter 118. In some examples the micromechanical resonator 150 is embedded in the sidewall 117 of the catheter 118. In other applications, the micromechanical resonator 150 is disposed on an interior of the sidewall 117 of the catheter 118 or disposed on an exterior of the sidewall 117 of the catheter 118. In other applications, the micromechanical resonator 150 can be orally consumed by the patent while being able to withstand and survive passing through the digestive tract of the patient. In some examples, the micromechanical resonator 150 can be embedded into the patient (e.g., via surgery).

In the example shown in FIG. 1, the patient 104 is laying on a bed 120 with a plurality of electromagnetic transducers 122 embedded in the bed 120. Each of the transducers 122 are connected to a computer system 108 and generally include one or more coils to produce and transmit an electro-magnetic field 166. Each transducer 122 is arranged to produce the electromagnetic field 166 in the vicinity of the patient 104. The transducers 122 can be configured to operate in both a transmit mode to transmit the electromagnetic field 166 and in a receive mode to receive an electro-magnetic field 167.

In some implementations, each transducer 122 produces a different electromagnetic field 166 so that distances and orientations between the micromechanical resonator 150 and the respective transducers 122 can be determined by the computer system 108. Each transducer 122 can also provide a different magnetic field. In some examples, the computer system 108 controls the transducers 122 by controlling them on and off so that only one of the transducers 122 is transmitting an electro-magnetic field 166 at a given time. While the transducers 122 are embedded in the bed 120 in this example, in some implementations, the transducers 122 are embedded elsewhere (e.g., in a C-arm machine).

When the transducers 122 are in receive mode, the transducers 122 sense a magnetic or electromagnetic signal 167 produced by the micromechanical resonator 150. The received (or sensed) magnetic or electromagnetic signal 167 is then processed by the computer system 108 to determine a position and/or orientation of the micromechanical resonator 150 within the anatomy of the patient 104.

In some cases, the computer system 108 controls a display 110 to present a representation of the determined position, orientation, etc. of the micromechanical resonator 150 within the anatomy of the patient 104. The computer system 108 includes one or more processors to determine spatial characteristics (e.g., the position and/or orientation) of the micromechanical resonator 150 within the anatomy of the patient 104 and control the display 110. The computer system 108 and/or medical professional 102 uses the spatial characteristics of the micromechanical resonator 150 within the anatomy of the patient 104 to determine the position and/or orientation of the catheter 118. This information can assist the medical professional 102 in navigating the catheter 118 through a tortuous anatomy of the patent 104 and determining when the catheter 118 has arrived at its destination within the patent 104. After use, the catheter 118 including the micromechanical resonators 150 can be disposed.

Preferably, the micromechanical resonator 150 is relatively small (e.g., typically on the order of millimeters (mm)). These dimensions allow the micromechanical resonator 150 to be embedded in medical instruments (e.g., the catheter 118) to assist in determining the position and/or orientation of the medical instrument. Further, the small nature of the micromechanical resonator 150 allows the catheter 118 to maintain a relatively small bending radius to assist in navigating the catheter 118 through the tortuous anatomy of the patent 104. As such, it can be preferable to taper one or more ends of the micromechanical resonator 150 to avoid limiting a bending radius of the medical instrument, for example. The micromechanical resonator 150 can also be used in a marker for tracking (e.g., to determine the position and/or orientation of the marker over time within the anatomy of the patient 104).

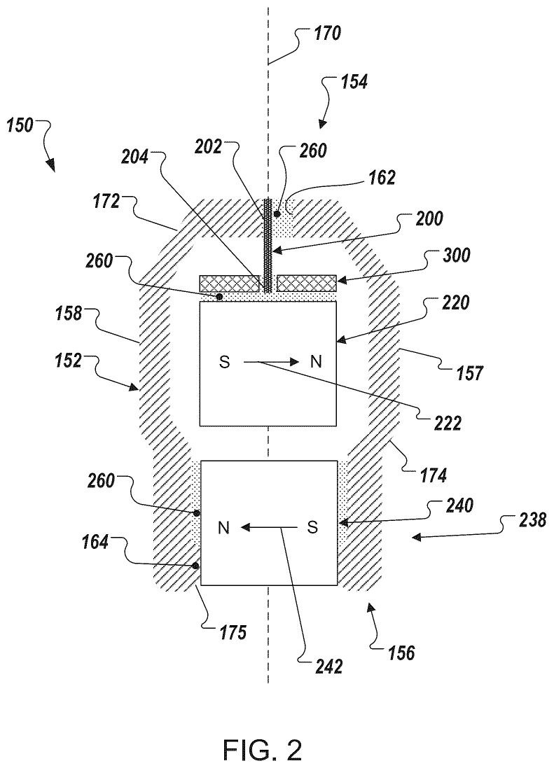

FIG. 2 shows an example micromechanical resonator 150 with two magnetic objects implemented as cylindrical permanent magnets. The micromechanical resonator 150 includes a housing 152 (e.g., an elongated housing, or casing) having a first end 154, a second end 156, and a sidewall 158 extending from the first end 154 to the second end 156. The housing 152 can be non-magnetic and substantially rigid (e.g., formed of plastic, 3D printed plastic, etc.). The housing 152 can be cylindrical having a diameter less than 2 mm (e.g., between 0.4 and 1.5 mm) and a length less than 4 mm (e.g., between 1 and 3 mm). The housing 152 can be 3D printed by a 2-photon polymerization technology. One or more ends of the housing 152 can be tapered. For example, the first end 154 can have a tapered portion 172. The housing 152 can be monolithic. The housing 152 can be a substantially monolithic structure.

The micromechanical resonator 150 includes a torsionally resilient slender member 200 (e.g., a filament, a fiber, a thread, a thread-like structure, a plurality of fibers, etc.) disposed in the housing 152 with a first end 202 of the filament 200 attached or fixed to the first end 154 of the housing 152 at the longitudinal center of the housing 152. The torsionally resilient slender member 200 can be cylindrical. The torsionally resilient slender member 200 will be referred to as a filament 200 throughout this disclosure but it should be understood that the torsionally resilient slender member 200 need not be limited to a filament 200.

The filament 200 can be or include a Kevlar® fiber, an ultra-high density polyethylene fiber, or a carbon fiber (e.g., a carbon nanofiber). The filament 200 can be or include one or more organic fibers in the aromatic polyamide family (aramids). For example, the filament 200 can include an aromatic polyamide (e.g., aramid) fiber. The filament 200 can be or include an aramid fiber (e.g., a Kevlar® aramid fiber).

Preferably, a torsional stiffness of the filament 200 is as small as possible (e.g., less than 1e-7 Nm/rad). A low torsional stiffness can limit the filament's 200 influence on torsional vibration modes. The torsional stiffness of the filament 200 depends on the size of the magnetic object that performs the rotational oscillation (e.g., the first magnetic object 220). It can be preferred to have the torsional stiffness of the filament 200 less than 1/10 of the stiffness due to the magnetic field. For example, for magnetic objects implemented as 0.5 mm diameter spheres the stiffness due to the magnetic field is about 1e-6 Nm/rad. This stiffness scales with the volume of the oscillating magnetic object. The torsional stiffness of the filament 200 can be reduced by using as thin of a filament 200 as possible.

Preferably, the axial stiffness of the filament 200 is as large as possible (e.g., greater than 1 GPa). Having a large axial stiffness can increase the likelihood of maintaining a constant spacing between two magnetic objects and the limit the likelihood of uniaxial vibration modes being excited during operation. In some cases, using materials with an axial stiffness between 10 GPa and 20 GPa is be preferred.

Preferably, the tensile strength of the filament 200 is as large as possible (e.g., greater than 1 GPa). Having a large tensile strength can decrease the likelihood of breakage of the filament 200 when the magnetic objects are forced towards one another. In some cases, materials having a tensile strength exceeding 1 GPa (e.g., exceeding 2 GPa) is be preferred. Since the torsionally resilient slender member 200 is thin, a high tensile strength is generally needed.

The bending stiffness of the filament 200 is typically not a design constraint. The inventors have found success in using materials with wide ranges of bending stiffnesses. The first end 154 of the filament 200 can be adhered (e.g., glued) to the housing 152 (e.g., by a cyanoacrylate, an epoxy-based adhesive, or a UV curable adhesive). The axial length of the filament 200 within the housing 152 is fixed. An axial length of the filament 200 inside the housing 152 can be between 300 and 800 μm. For example, the axial length of the filament 200 inside the housing 152 can be between 300 and 500 μm. A diameter of the filament 200 can be between 4 and 20 μm. For example, the diameter of the filament 200 can be between 4 and 12 μm.

The micromechanical resonator 150 includes a first magnetic object 220 (e.g., a first permanent magnet or coil) disposed in the housing 152 with a second end 204 of the filament 200 connected (e.g., directly or indirectly attached or fixed) to the first magnetic object 220 via a first part 300 (e.g., an attachment part, an alignment part, etc.). The second end 204 of the filament 200 can be adhered (e.g., glued) to the first part 300 and/or the first magnetic object 220 by an adhesive 260 (e.g., by a cyanoacrylate, an epoxy-based adhesive, or a UV curable adhesive). A magnetic axis 222 of the first magnetic object 220 is perpendicular to a longitudinal axis 170 of the housing 152, with a north and south pole oriented perpendicular to the longitudinal axis 170. The first magnetic object 220 has a magnetic dipole. The first magnetic object 220 can be a permanent magnet. The first magnetic object 220 can be a cylindrical permanent magnet with the second end 204 of the filament 200 connected to an axial end of the permanent magnet. The first part 300 can distribute the loading between the filament 200 and the first magnetic object 220 and align the first magnetic object 220 at the longitudinal center of the housing 152.

The micromechanical resonator 150 includes a restoring torque unit 238 disposed in the housing 152 and being spaced apart from the first magnetic object 220. The restoring torque unit 238 can be a second magnetic object 240 (e.g., a second permanent magnet, coil, etc.). The second magnetic object 240 can be fixed relative to the housing 152, e.g., by an adhesive 260. The adhesive 260 can be a cyanoacrylate, an epoxy-based adhesive, or a UV curable adhesive. A magnetic axis 242 of the second magnetic object 240 is perpendicular to the longitudinal axis 170 of the housing 152, with a north and south pole oriented perpendicular to the longitudinal axis 170. The second magnetic object 240 has a magnetic dipole. The magnetic axis 222 of the first magnetic object 220 and the magnetic axis 242 of the second magnetic object 240 have opposite polarities (e.g., the respective north and south poles are oriented in opposite directions to one another). In this way, the magnetic axis 242 of the second magnetic object 240 is anti-parallel to the magnetic axis 222 of the first magnetic object 220. The second magnetic object 240 can be a spherical or cylindrical permanent magnet having a larger diameter than the first magnetic object 220.

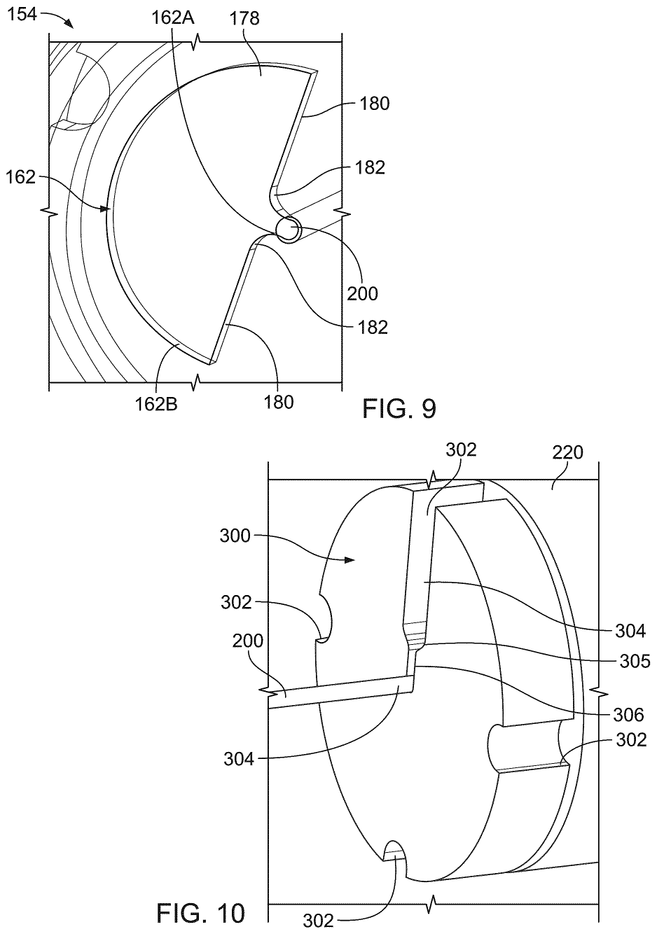

The housing 152 includes a first opening 162 at the first end 154 of the housing 152. The first opening 162 includes a groove 162A at a longitudinal center of the housing 152 (see, e.g., FIGS. 8 and 9) and an enlarged opening 162B that is open to the groove 162A (see, e.g., FIGS. 8 and 9). The enlarged opening 162B is sized to allow axial insertion of the filament through the first end of the housing 152 (e.g., under suction from a vacuum) and then allow the filament 200 to be laterally moved into the groove 162A. Once assembled, the first end 202 of the filament 200 is disposed within the groove 162A and adhered to the sidewall of the groove 162A by an adhesive, and the filament 200 is located at the longitudinal center of the housing 152.

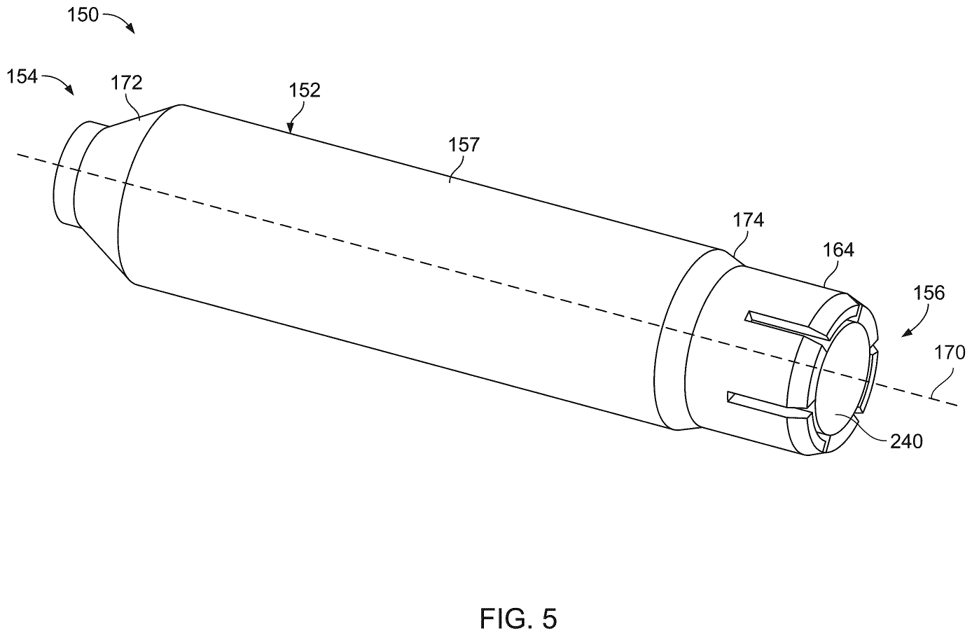

The housing 152 includes one or more resilient arms 164 disposed at the second end 156 of the housing 152 (see, e.g., FIG. 5). The one or more resilient arms 164 apply a radially inward force to the second magnetic object 240 for holding the second magnetic object 240 in a fixed location within the housing 152. The one or more resilient arms 164 have a tapered portion 175 (e.g., a tapered internal diameter) to allow outward radial deflection of the one or more resilient arms 164 when the second magnetic object 240 is inserted into the housing 152 during assembly. The outward radial deflection of the one or more resilient arms 164 causes the radially inward force on the second magnetic object 240 to be applied due to a spring-back resiliency of the one or more resilient arms 164 that forces the one or more resilient arms 164 to their undeflected position.

The housing 152 includes a recessed area 176 (e.g., a reservoir) disposed at the first end 154 of the housing 152 (see, e.g., FIGS. 8 and 9). The recessed area 176 can be for adhesive to collect when adhering the filament 200 to the housing 152.

In equilibrium, the first and second magnetic objects 220, 240 align with anti-parallel orientation of their magnetization. An external magnetic or electromagnetic field pulse can be used to start a mechanical resonance via a rotational oscillation of the first magnetic object 220 relative to the second magnetic object 240. The filament 200 resiliently deforms (e.g., twists) by torsion to allow the first magnetic object 220 to oscillate relative to the second magnetic object 240 in response to the external magnetic or electromagnetic field pulse.

The first magnetic object 220 can rotate out of the equilibrium orientation in response to the external magnetic or electromagnetic field pulse to initiate rotational oscillations relative to the second magnetic object 240. The first magnetic object 220 is configured to rotate (e.g., oscillate) about the longitudinal axis 170 of the housing 152 relative to the second magnetic object 240 while maintaining a fixed distance to the second magnetic object 240 in response to the external magnetic or electromagnetic field pulse. The first magnetic object 220 is suspended in the housing 152 by the filament 200 and preferably does not contact the sidewall 158 of the housing 152 during oscillations relative to the second magnetic object 240 in response to the external magnetic field pulse. Preferably, the first magnetic object 220 oscillates amount an angle greater than 20 degrees relative to the second magnetic object 240. Importantly, the micromechanical resonator 150 does not require electronics to produce the oscillations and is completely passive.

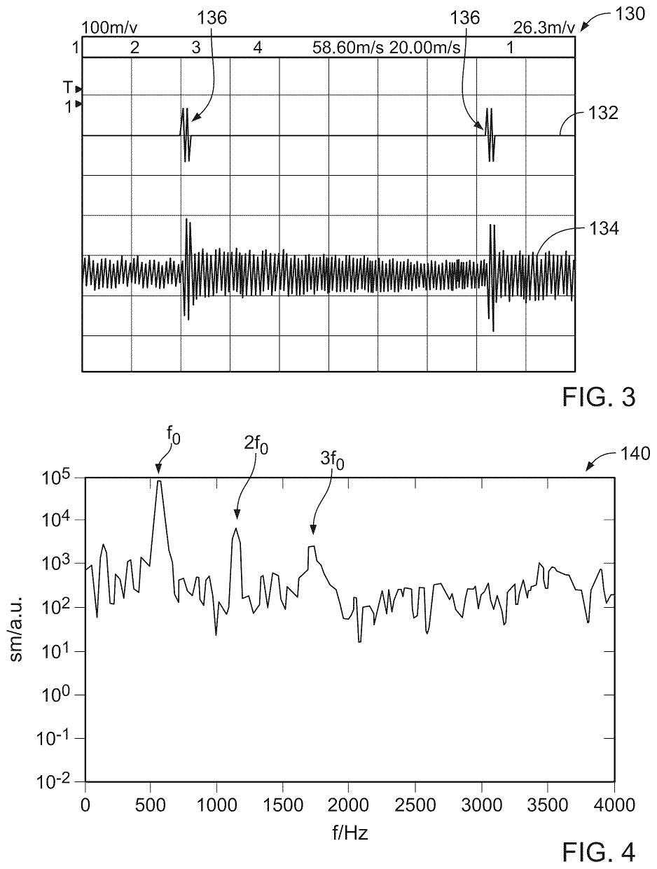

FIG. 3 is a time-domain plot 130 illustrating voltages of the external magnetic or electromagnetic field 166. The one or more processors of the computer system 108 generate transmit pulses (e.g., upper signal 132 in FIG. 3) that are amplified using an audio amplifier and then sent to transmit coils of the transducers 122. FIG. 3 shows, besides the transmit pulses 132, the induced voltage 134 in a receive coil of the transducers 122. The spacing of the excitation pulses 136 can be continuously adjusted by the one or more processors of the computer system 108. Once the transducers 122 measure the time-domain induced voltage 134 representing the magnetic field 167, the computer system 108 receives the induced voltage 134 and performs a Fast Fourier Transform (FFT) on the time-domain signal induced voltage 134 to produce a frequency-domain signal representing the magnetic field 167.

FIG. 4 is a frequency-domain plot 140 illustrating example detected resonances of the micromechanical resonators. The one or more resonance frequencies of the oscillating magnet are detectable by the computer system 108. Specifically, the fundamental frequency of oscillation of the first magnetic object 220 relative to the second magnetic object 240 is represented as f₀, with second and higher harmonics represented as 2f₀, 3f₀, etc. In some implementations, the computer system 108 determines the magnitude of the peaks in the time-domain or frequency-domain and uses these peaks to determine the position and/or orientation of the micromechanical resonator 150.

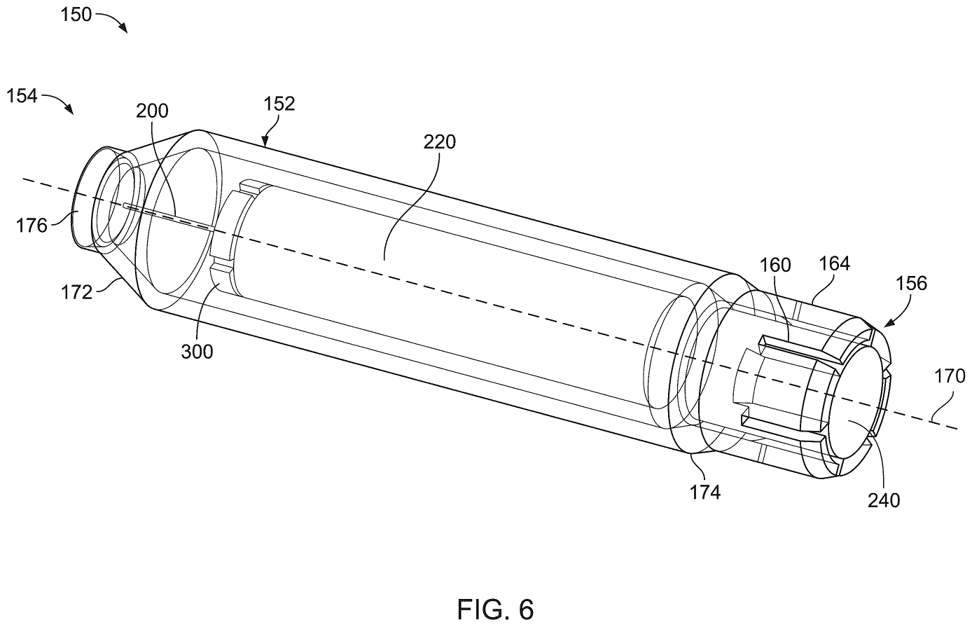

FIGS. 5 and 6 show the micromechanical resonator 150 in further detail. The housing 152 includes a cylindrical portion. The first end 154 of the housing 152 and the second end 156 of the housing 152 are tapered toward the longitudinal axis 170 to reduce the external diameter of the micromechanical resonator 150 at each axial end 154, 156 of the housing 152. A total axial length of the housing 152 can be less than 20 mm (e.g., between 2 and 5 mm, less than 10 mm, etc.). The first end 154 of the housing 152 has a first tapered portion 172 that is tapered relative to a cylindrical portion 157 of the housing 152. The second end 156 of the housing 152 has a second tapered portion 174 that is tapered relative to the cylindrical portion 157 of the housing 152. The first and second tapered portions 172, 174 extend directly from the cylindrical portion 157.

The first end 154 is configured for filament 200 feedthrough and center alignment of the filament 200. The first end 154 can be shaped for a longer filament 200 without having a longer housing of a full diameter due to the tapered wall geometry of the first tapered portion 172. The first end 154 includes a reservoir for adhesive.

The second end 156 includes a clamping structure 164 (e.g., a resilient arm, a set screw, a clamp, etc.) to guide and hold the second magnetic object 240 within the housing 152. The clamping structure 164 will be referred to as one or more resilient arms 164 throughout this disclosure but it should be understood that the clamping structure 164 need not be limited to resilient arms. The one or more resilient arms 164 can extend in a longitudinal direction and surround the second permanent magnet 240. The one or more resilient arms 164 can be integrally formed with the housing 152. As described above, the one or more resilient arms 164 apply a radially inward force to the second magnetic object 240 for holding the second magnetic object 240 in a fixed location within the housing 152. Openings 160 (e.g., circumferential openings, slits, etc.) between each respective resilient arm of the one or more resilient arms 164 allows adhesive to be inserted through the openings 160 to improve dispersion of the adhesive and improve contact area between the one or more resilient arms 164 and the second magnetic object 240.

The micromechanical resonator 150 is configured such that during assembly and prior to the second magnetic object 240 being adhered to the one or more resilient arms 164, the one or more resilient arms 164 (i) allow the second magnetic object 240 to move axially relative to the housing 152 when an axial force exerted on the second magnetic object 240 exceeds a threshold force and (ii) hold the second magnetic object 240 axially relative to the housing 152 when the axial force exerted on the second magnetic object 240 is below the threshold force. The micromechanical resonator 150 is configured such that during assembly and prior to the second magnetic object 240 being adhered to the one or more resilient arms 164, an axial movement of the second magnetic object 240 relative to the housing 152 changes a resonant frequency in which the first magnetic object 220 oscillates about the longitudinal axis 170 of the housing 152 relative to the second magnetic object 220.

The second magnetic object 240 can have an axial length of less than 2 mm (e.g., less than 1 mm, e.g., 0.7 mm). The first magnetic object 220 can have an axial length that is longer than the axial length of the second magnetic object 240. The axial length of the second magnetic object 240 can be greater than 0.5 mm (e.g., greater than 1 mm, e.g., 2 mm).

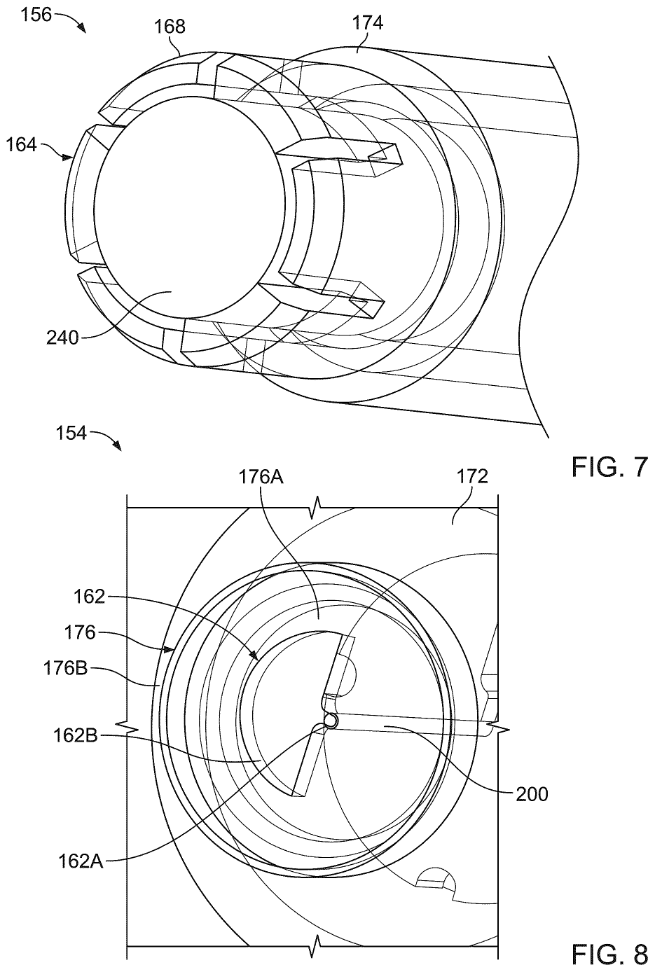

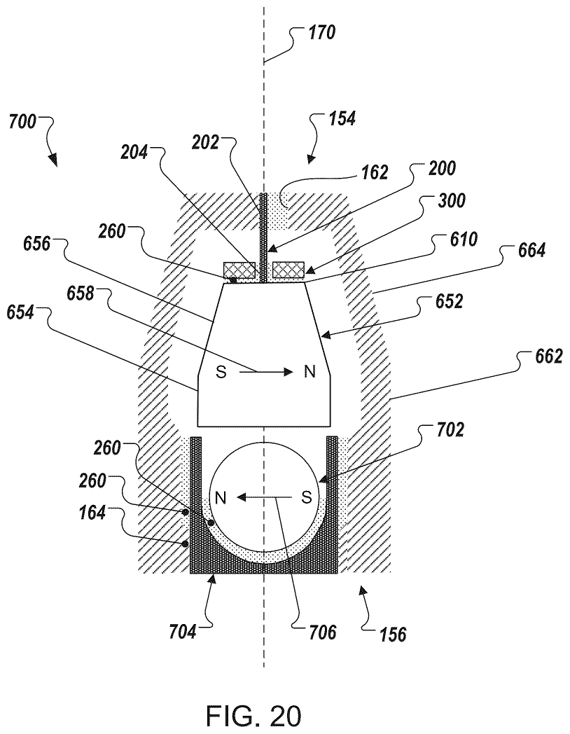

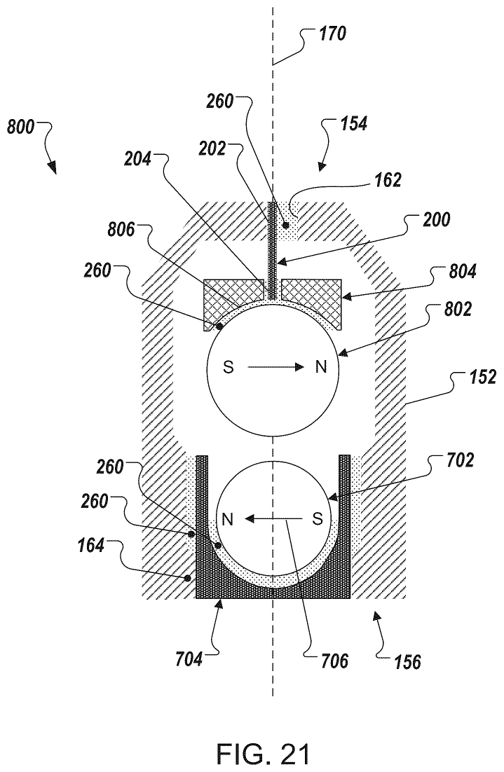

FIG. 7 is a close-up view of the second end 156 of the housing 152. The second end 156 can include one or more resilient arms 164 circumferentially spaced around the longitudinal axis 170. The second end 156 can include three resilient arms 164. The second end 156 can include more or less than three arms (e.g., one arm, two arms, four arms, six arms, ten arms, etc.). The one or more resilient arms 164 can extend axially from the second tapered portion 174 of the housing 152. The one or more resilient arms 164 frictionally engage (e.g., via an interference fit) an outer diameter of the second magnet 240 (or a second part 704 (e.g., a carrier)-see, e.g., FIGS. 20 and 21).

One or more rigid portions 168 of the housing 152 can be disposed between respective pairs of the one or more resilient arms 164. In the implementation shown, three rigid portions 168 are disposed circumferentially between three resilient arms 164, although other implementations can include more (e.g., greater than three (e.g., between three and ten)) or less (e.g., zero, one, or two) resilient arms and/or rigid portions. The one or more resilient arms 164 can extend further radially inward from the housing 152 than the rigid portions 168. The one or more resilient arms 164 and/or the rigid portions 169 can be integrally formed with the housing 152. The one or more resilient arms 164 and/or the rigid portions 169 can be printed with the housing 152 using a 2-photon polymerization technology 3D printing technique.

FIG. 8 is a close-up view of the first end 154 of the housing 152. The recessed area 176 can be defined by a bottom surface 176A and a sidewall 176B that surrounds the bottom surface 176A. The bottom surface 176A can be defined by the housing 152 and located at the first tapered portion 172 of the housing 152. The sidewall 176B can extend in a longitudinal direction from the tapered portion 172 of the housing 152. The bottom surface 176A of the recessed area 176 can define the first opening 162 at the first end 154 of the housing 152. For example, the groove 162A and the enlarged opening 162B defining the first opening 162 can be a cutout in the bottom surface 176A. As noted above, the enlarged opening 162B can be open to the groove 162A such that during assembly, the filament 200 can be axially inserted through the enlarged opening 162B, then laterally moved into the groove 162A, and then adhered to the sidewalls of the groove 162A to fix the filament 200 to the housing 152. When adhesive is applied, the adhesive can pool in the recessed area 176 and substantially fill the recessed area 176. Thus, in some examples, cured adhesive can completely cover the groove 162A and the enlarged opening 162B of the first opening 162 when the filament 200 is adhered to the housing 152 within the groove 162A.

FIG. 9 is a close-up view of the first end 154 of the housing 152 showing further detail of the first opening 162. A first lateral side of the groove 162A can be open to the enlarged opening 162B while a second lateral side of the groove 162A is not open to the enlarged opening 162B. The enlarged opening 162B can be disposed on a single lateral side of the groove 162A. The enlarged opening 162B can have a semi-circular shape. However, other shapes can be used in other implementations (e.g., square-shaped openings or other polygonal-shaped openings). The enlarged opening 162B can have a first sidewall 178 that is curved (e.g., semi-circular) and a second sidewall 180 that is planar. The sidewalls 178, 180 can be tapered to help guide the filament 200 into the groove 162A.

The groove 162A can be recessed into the second sidewall 180. The groove 162A can be recessed into the second sidewall 180 at the center of the second sidewall 180. A transition region 182 between the groove 162A and the enlarged opening 162B can be curved or angled to guide the filament 200 into the groove 162A during assembly.

FIG. 10 is a close-up view of the first part 300 (e.g., the attachment part, the alignment part, etc.). The first part 300 can be or include a plate. The first part 300 is attached to the first end of the first magnetic object 220 and to the second end 204 of the filament 200. As described above, the first part 300 can distribute the adherence between the filament 200 and the first magnetic object 220 to increase the likelihood of the filament 200 being securely adhered to the first magnetic object 220. The first part 500 can concentrically align the first magnetic object 220 to the housing 152. An outer diameter of the first part 500 can substantially correspond to (e.g., match) an outer diameter of the first magnetic object 220. Having the outer diameters substantially correspond to one another can be helpful for centering the first part 500 on the first magnetic object 220.

The first part 300 includes one or more openings 302 extending through the first part 300. The one or more openings 302 can extend through the first part 300 in a longitudinal direction of the first part 300. The one or more openings 302 can include two or more openings (e.g., three, four, five, etc.) that are spaced apart from one another around a longitudinal axis of the first part 300. The one or more openings 302 can allow adhesive to be applied to the space between the first part 300 and the first magnetic object 220 during assembly. The one or more openings 302 can allow for lateral insertion of the filament 200 on the first part 300.

The filament 200 can extend through at least one opening 302 of the one or more openings 302. The filament 200 can be adhered to sidewalls of the first part 300 defining the at least one opening 302 of the one or more openings 302. The at least one opening 302 can be located at a longitudinal axis of the first part 300. Having the filament 200 extending through the at least one opening 302 at the longitudinal axis of the first part 300 can centrally align the filament 200 to the longitudinal axis of the housing 152.

The one or more openings 302 can include a wide opening 304 and a narrow opening 306 that are open to one another. The wide opening 304 can transition to the narrow opening 306 at a transition region 508. The transition region 305 can help guide the filament 200 from the wide opening 304 to the narrow opening 306 during assembly. The narrow opening 306 can be located at the longitudinal axis of the first part 300. The wide opening 304 can be open at an outer perimeter (e.g., outer diameter) of the first part for allowing the filament 200 to be laterally inserted into the wide opening 304 during assembly.

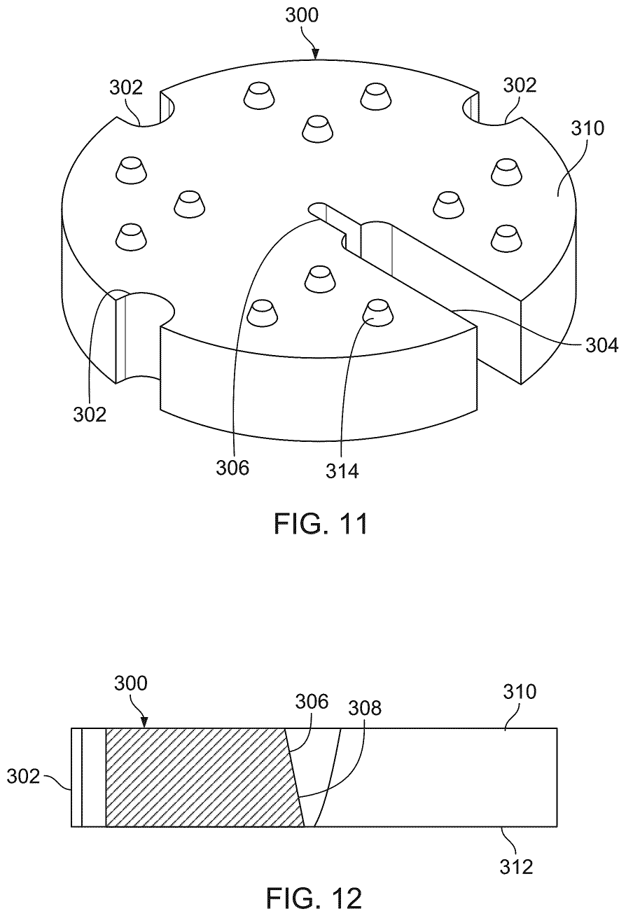

FIGS. 11 and 12 are views of the first part 300 in isolation. The narrow opening 306 at the longitudinal center of the first part 300 has a tapered wall 308 extending from a first side 310 of the first part 300 to a second side 312 of the first part 300. The tapered wall 308 can be tapered toward the longitudinal axis of the first part 300 such that a distance from the tapered wall 308 to the longitudinal axis of the first part 300 decreases in a longitudinal direction from the first side 310 to second side 312. The first side 310 can face toward the first end 154 of the housing 152 and the second side 312 can face toward the first magnetic object 220.

The narrow opening 306 at the second side 312 of the first part 300 can be used to align the filament 200 to the longitudinal center of the first part 300- and thus to the longitudinal center of the first magnetic object 220. The opening 306 at the first side 310 of the first part 300 is angled away from the longitudinal axis to reduce the likelihood of the filament 200 contacting the tapered wall 308 at the first side 310. The filament 200 is preferably only glued/fixed at the second side 312. By not contact the first part 300 at the first side 310, the tapered wall 308 effectively extends the “free length” of the filament 200 in which the filament 200 does not contact another part. Extending the free length increases the resonance quality. Thus, it can be preferred to have as long of a filament 200 as possible. The wall 318 can be cone-shaped.

The first part 300 can include a plurality of distance elements 314 (e.g., axially-extending protrusions, standoffs, etc.) integrally formed with the first part 300. The distance elements 314 separate a remainder of the first part 300 from the first magnetic object 220. The distance elements 314 can help to increase the likelihood that the first part 300 is in parallel alignment to a surface of the first magnetic object 220 in contact with the first part 300. The distance elements 314 are configured to allow the first part 300 to be substantially parallel to the first magnetic object 220 when the first part 300 is in contact with the first magnetic object 220.

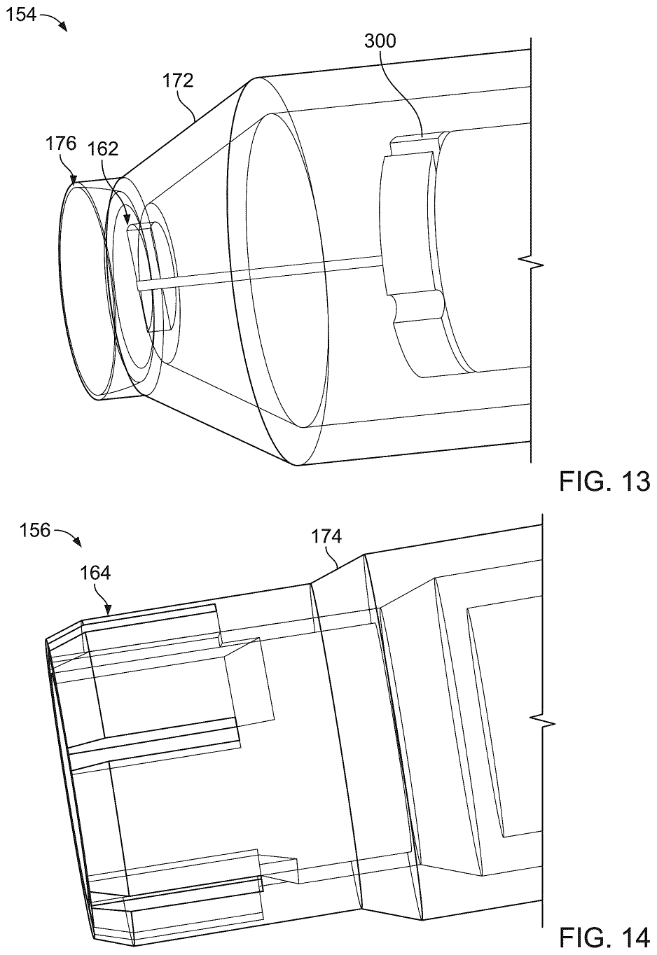

The distance elements 314 can provide axial clearance for the filament 200 to cross perpendicularly one or more times between the first part 300 and the first magnetic object 220 without angling or tilting the first part 300 relative to the first magnetic object 220. The axial clearance between the first part 300 and the first magnetic object 220 defined by the distance elements 314 can be filled with adhesive. FIG. 13 is a close-up view of the first end 154 of the housing 152 showing additional detail of the recessed area 176, the first opening 162, and the first tapered portion 172. FIG. 14 is a close-up view of the second end 156 of the housing 152 showing additional detail of the second tapered portion 174, and the resilient arms 164. In some examples, the size of the taper, and the first and second ends 154, 156, can be extended for a longer filament lengths so that the full housing diameter is not needed for the entire housing length.

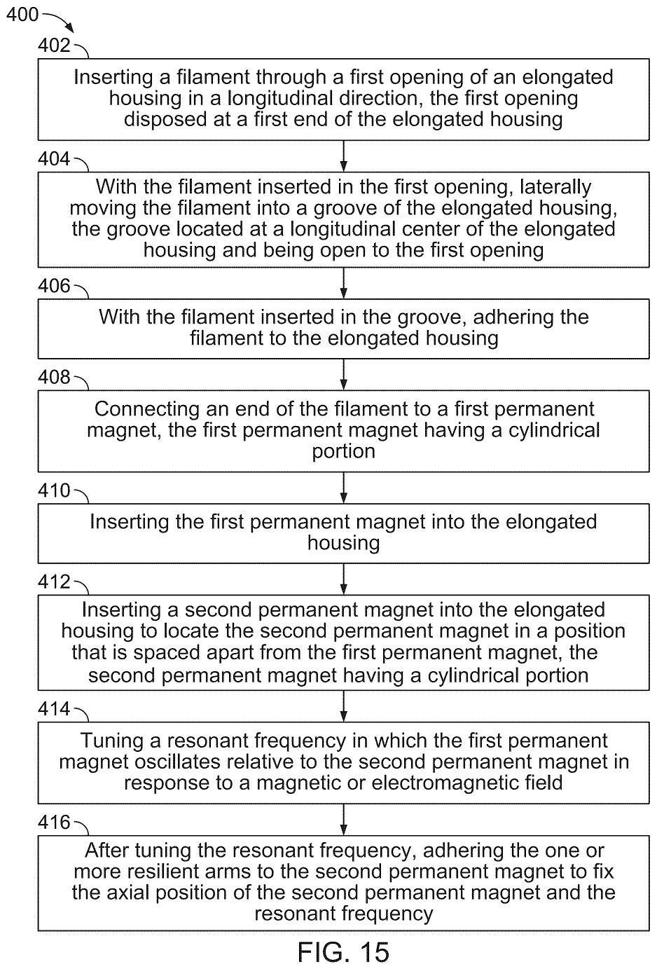

FIG. 15 is an example method 400 for assembling the micromechanical resonator 150. At step 402, the method 400 includes inserting a filament through a first opening of an elongated housing in a longitudinal direction, the first opening disposed at a first end of the elongated housing. For example, referring to FIG. 2, the filament 200 is inserted through the first opening 162 disposed at the first end 154 of the housing 152.

At step 404, the method 400 includes with the filament inserted in the first opening, laterally moving the filament into a groove of the elongated housing, the groove located at a longitudinal center of the elongated housing and being open to the first opening. For example, referring to FIG. 8, the filament 200 is inserted through the enlarged opening 162B of the first opening 162 and then laterally moved into the groove 162A. The groove 162A being located at a longitudinal center of the housing 152 and being open to the enlarged opening 162B.

At step 406, the method 400 includes with the filament inserted in the groove, adhering the filament to the elongated housing. For example, referring to FIG. 2, the filament 200 is adhered to the housing 152 using an adhesive located within the groove 162A. The adhesive can be a cyanoacrylate or epoxy-based adhesive.

At step 408, the method 400 includes connecting an end of the filament to a first permanent magnet, the first permanent magnet having a cylindrical portion. For example, referring to FIG. 2, the end 204 of the filament 200 is connected to the first magnetic object 220 via the first part 300. The first magnetic object 220 can be a permanent magnet having a cylindrical portion (e.g., be completely cylindrical or include a tapered cylinder portion).

At step 410, the method 400 includes inserting the first permanent magnet into the elongated housing. For example, referring to FIG. 2, the first magnetic object 220 is inserted into the housing 152 (e.g., by axially sliding the first magnetic object 220 with the filament 200 connected thereto through an opening at the second end 156 of the housing 152).

At step 412, the method 400 includes inserting a second permanent magnet into the elongated housing to locate the second permanent magnet in a position that is spaced apart from the first magnet, the second permanent magnet having a cylindrical portion. For example, referring to FIG. 2, the second magnetic object 240 is inserted into the housing 152 (e.g., by axially sliding the second magnetic object 240 through the opening at the second end 156 of the housing 152). The second magnetic object 240 can be a permanent magnet having a cylindrical portion (e.g., be completely cylindrical or include a tapered cylinder portion). The second magnetic object 240 can be spaced apart (e.g., axially spaced apart) from the first magnetic object 220.

At step 414, the method 400 includes tuning a resonant frequency in which the first permanent magnet oscillates relative to the second permanent magnet in response to a magnetic or electromagnetic field. For example, referring to FIG. 2, a resonant frequency in which the first magnetic object 220 oscillates relative to the second magnetic object 240 can be tuned by varying an axial position of the second magnetic object 240 within the housing 152 before the second magnetic object 240 is adhered to the housing 152.

The resonant frequency will depend on the size of the first and second magnetic objects 220, 240, the spacing between them, and the mechanical properties of the filament 200. Controlling the axial position of the second magnetic object 240 within the housing 152 can directly affect the resonance frequency in which the first magnetic object 220 oscillates in response to an external magnetic or electromagnetic field.

In some examples, the axial position of the second magnetic object 240 is varied by applying an axial force to the second magnetic object 240 while one or more resilient arms 164 of the housing 152 apply a radially inward force to the second magnetic object 240. In some examples, the resonant frequency is tuned by varying the axial position of the second magnetic object 240 within the housing 152 while the filament 200 is adhered to the groove 162A.

At step 416, the method 400 includes after tuning the resonant frequency, adhering the one or more resilient arms to the second permanent magnet to fix the axial position of the second permanent magnet and the resonant frequency. For example, referring to FIG. 2, the one or more resilient arms 164 are adhered to the second magnetic object 240 by an adhesive 260 to fix the axial position of the second magnetic object 240 and the resonant frequency.

In addition, the axial spacing between the first magnetic object 220 and the second magnetic object 240 remains fixed after the filament 200 has been adhered to the housing 152 and the second magnetic object 240 has been adhered to the housing 152. In some examples, the housing 512 has been printed using 2-photon polymerization technology.

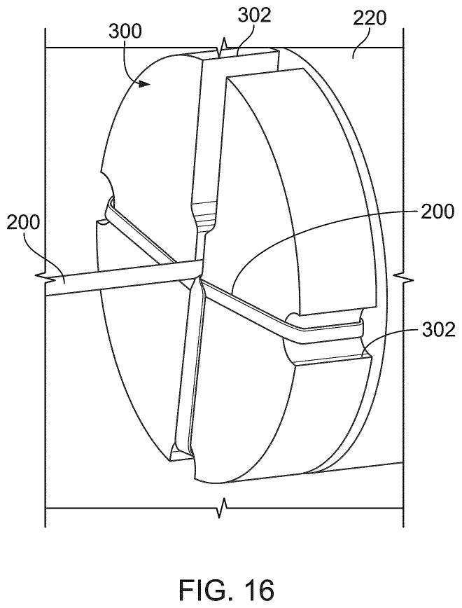

FIG. 16 shows a variation in which the filament 200 is at least partially wound around the first part 300. As described with reference to FIG. 10, the one or more openings 302 can include two or more openings (e.g., three, four, five, etc.) that are spaced apart from one another around a longitudinal axis of the first part 300. In the example shown in FIG. 16, the filament 200 extends through at least two of the openings 302 to provide an increased grip of the filament 200 on the first part 300. The filament 200 can be inserted onto the first part 300 in substantially the same manner as described with reference to FIG. 10.

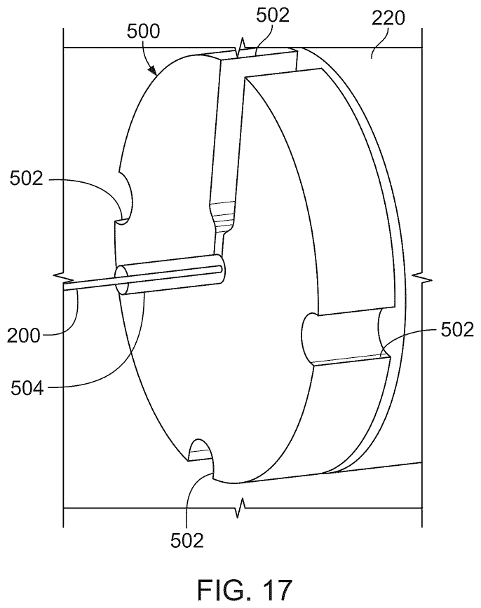

FIG. 17 shows a variation in which the first part has a protrusion to secure the filament 200 to the first part. In FIG. 17, a first part 500 has one or more openings 502 and an axially-extending protrusion 504. The one or more openings 502 can be substantially the same as the first part 300 for allowing adhesive to be inserted between the first part 500 and the first magnet object 220. The protrusion 504 can be located at the longitudinal center of the first part 500. The filament 200 can be attached (e.g., adhered) to a side of the protrusion 504. The filament 200 can be disposed within a cavity of the protrusion 504 and adhered to a sidewall of the cavity.

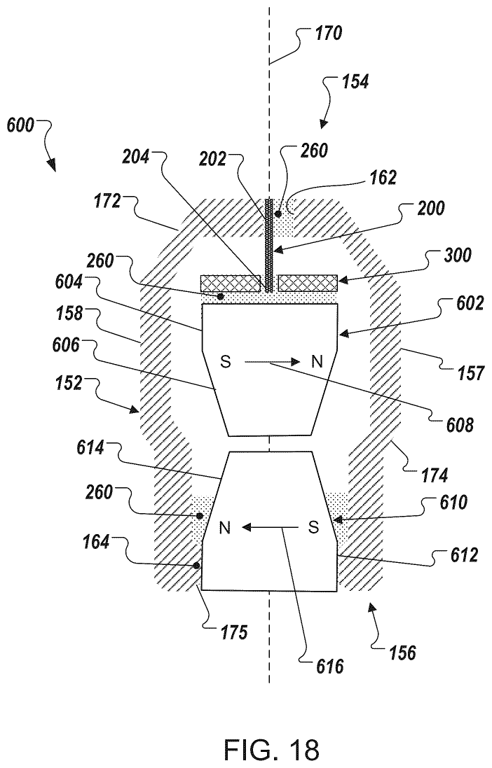

FIG. 18 shows a micromechanical resonator 600 that is substantially similar to the micromechanical resonator 150 except that a first magnetic object 602 includes a cylindrical portion 604 and a tapered cylindrical portion 606, and a second magnetic object 610 includes a cylindrical portion 612 and a tapered cylindrical portion 614.

The first magnetic object 602 and/or the second magnetic object 610 can be one or more permanent magnets or coils. A magnetic axis 608 of the first magnetic object 602 extends in a first direction perpendicular to a longitudinal axis of the first magnetic object 602, and a magnetic axis 616 of the second magnetic object 610 extends in a direction opposite to the first magnetic axis 608. The tapered cylindrical portion 606 of the first magnetic object 602 has a tapered end towards the second magnetic object 610, and the tapered cylindrical portion 614 of the second magnetic object 610 has a tapered end towards the first magnetic object 602. The tapered cylindrical portion 604 and/or the tapered cylindrical portion 614 can be frustoconical. The tapered cylindrical portion 604 and the tapered cylindrical portion 614 can directly face toward one another. In some implementations, only one of the first or second magnetic objects can include a tapered cylindrical portion and the other of the first or second magnetic objects can be entirely cylindrical (e.g., not having a tapered cylindrical portion). In some examples, the second magnetic object has a tapered cylindrical portion while the first magnetic object entirely cylindrical.

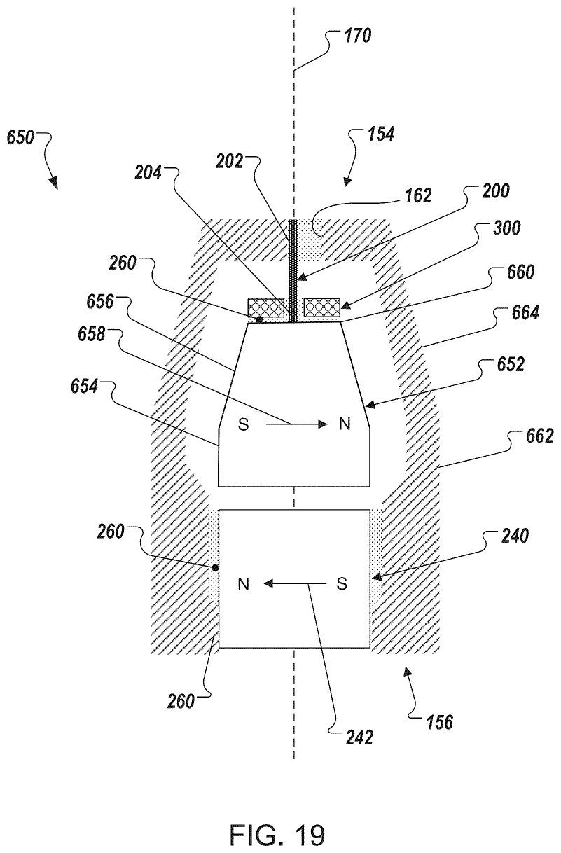

FIG. 19 shows a micromechanical resonator 650 that is substantially similar to the micromechanical resonator 150 except that a first magnetic object 652 includes a cylindrical portion 654 and a tapered cylindrical portion 656 and a housing 662 that includes a longer tapered portion 664 than the housing 152. The first magnetic object 652 can be a permanent magnet or coil. A magnetic axis 658 extends in a direction perpendicular to a longitudinal axis of the first magnetic object 652. The tapered cylindrical portion 654 has a tapered end 660 away from the second magnetic object 240. The tapered cylindrical portion 656 can be frustoconical. Having a first magnetic object with a tapered cylindrical portion that faces away from the second magnetic object can allow the housing to be more compact for allowing the micromechanical resonator 650 to fit in tighter spaces.

FIG. 20 shows a micromechanical resonator 700 that is substantially similar to the micromechanical resonator 650 except that a second magnetic object 702 is spherical and disposed within a second part 704 (e.g., a retainer or carrier). The second magnetic object 702 can be a permanent magnet or coil. When the second part is 704 is used, the second magnetic object 702 may not directly contact the resilient arms 164 of the housing 152. Instead, the second part 704 can slidably engage the resilient arms 164 during assembly and resonant frequency tuning and be adhered directly to the resilient arms 164 (e.g., using the adhesive 260). The second magnetic object 702 disposed within the second part 704 can be adhered to the second part 704 (e.g., using the adhesive 260) to limit relative movement between the second part 704 and the second magnetic object 702. The second part 704 can be helpful for securing non-cylindrical magnetic objects to the housings of the micromechanical resonators. The second part 704 can be used with any of the housings disclosed herein.

The second part 704 can be used to support the second magnetic object 702 and position the second magnetic object 702 at a fixed axial distance from the first magnetic object 652. The second part 704 can be inserted into the housing 662 from the second end 156. An adhesive 260 (e.g., a cyanoacrylate, an epoxy-based adhesive, or a UV curable adhesive) can be applied between the second part 704 and the housing 662 to fix the second part 704 and thus the second magnetic object 702 relative to the housing 662. The second part 704 centers the second magnetic object 702 radially within the housing 662. The second part 704 can be 3D printed using a 2-photon polymerization technology.

FIG. 21 shows a micromechanical resonator 800 with two spherical magnetic objects—a first spherical magnetic object 802 and the second spherical magnetic object 702 described with reference to FIG. 20. The second spherical magnetic object 702 is disposed in the second part 704 as described with reference to FIG. 20. The first spherical magnetic object 802 can be a permanent magnet or coil. The first spherical magnetic object 802 is connected to the filament 200 by a first part 804 (e.g., an attachment part).

The first part 804 is similar to the first part 300 except that the first part 804 has a curved recessed portion 806 that is curved to substantially match the shape or contour of the first spherical magnetic object 802. Adhesive 260 is disposed between the first part 804 and the first spherical magnetic object 802 and the filament 200 is attached through a central opening of the first part 804. Having a first part 804 with a curved recessed portion 806 that is curved to substantially match the shape or contour of the first spherical magnetic object 802 can provide improved adhesion between the first part 804 and the first spherical magnetic object 802 and thus provide a stronger connection between the filament 200 and the first spherical magnetic object 802.

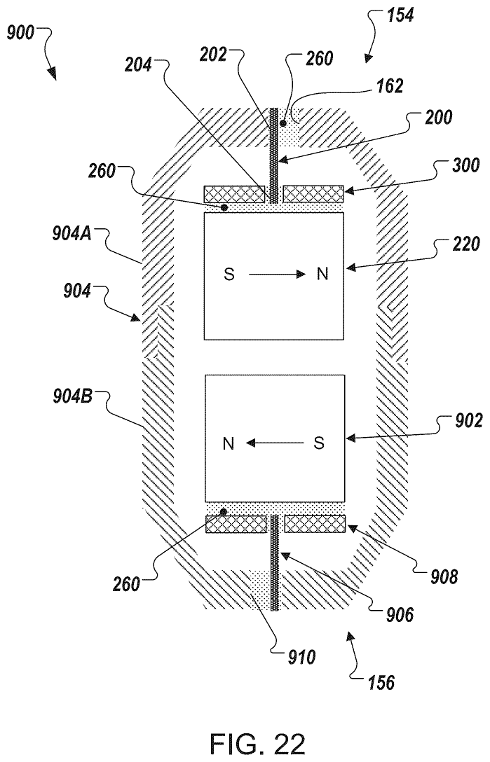

FIG. 22 shows a micromechanical resonator 900 with two movable magnetic objects. Both the first magnetic object 220 and a second magnetic object 902 can freely oscillate about a longitudinal axis of the housing 904 and relative to one another. Thus, in this embodiment, the housing 904 may not have resilient arms 164 since the second magnetic object 902 is not fixedly attached to the housing 904. Instead, the housing 904 can be substantially symmetric about a plane passing through a longitudinal center of the housing 904 that is perpendicular to a longitudinal axis of the housing 904. For assembly, the housing 904 includes a first portion 904A and a second portion 904B that engage and align with one another. The first portion 904A can be adhered to the second portion 904B using an adhesive.

The second magnetic object 902 is connected to a second filament 906 that is substantially the same as the first filament 200. The second magnetic object 902 can be attached to a second, first part 908 that is substantially the same as the first, first part 300. The housing 904 can have a second opening 910 at the second end 156 that is substantially the same as the first opening 162.

An example method for manufacturing the housing 152 can include printing the housing using a 3D printing technology. The housing 152 can be printed to include the first opening 162 located at the first end 154 of the housing 152 directly. The housing 152 can be printed to include the one or more axially-extending resilient arms 164 located at the second end 156 of the housing 152 directly. The housing 152 can be printed such that the first opening 162 includes the groove 162A recessed relative to the enlarged portion 162B and open to the enlarged portion 162B. The groove 162A being located at a longitudinal center of the housing 152, and the enlarged opening 162B having a semi-circular or other polygonal shape.

In some examples, the housing 152 is printed such that a longitudinal length of the housing 152 is less than 10 mm. In some examples, the longitudinal length of the housing is between 2 and 5 mm. In some examples, the 3D printing technology is a 2-photon polymerization technology. A 2-photon polymerization technology can provide precise printing of features with a resolution down to 200 nm and can print the housing 152 having a diameter of less than 2 mm (e.g., between 0.4 and 1.5 mm) and a length of less than 10 mm (e.g., between 2 and 5 mm).

In some examples, the housing 152 is printed such that the first end 154 of the housing 152 is tapered relative to a cylindrical portion 157 of the housing 152. In some examples, the housing 152 is printed such that the second end 156 of the housing 152 is tapered relative to the cylindrical portion 157 of the housing 152.

A system can include one or more micromechanical resonators 150. Each micromechanical resonator 150 can be configured to have a different resonant frequency. For example, different resonant frequencies can result from different axial spacing between the respective first and second magnetic objects 220, 240 of the one or more micromechanical resonators 150. For example, different axial positions of the second magnetic object 240 within the housing 152 can provide different axial spacings between the respective first and second magnetic objects 220, 240 which can be used to control the resonant frequency in which the first magnetic object 220 oscillates in response to an external magnetic or electromagnetic field.

In some examples, each first magnetic object 220 can be a first permanent magnet disposed in the housing 152 and being connected to a second end of a filament 200. Each first permanent magnet can have a cylindrical portion. In some examples, each second magnetic object 240 can be a second permanent magnet disposed in the housing 152 and being attached to the housing 152 at a second end 156 of the housing 152. Each second permanent magnet can have a cylindrical portion and be axially spaced apart from each respective first permanent magnet. The filament 200 can be configured to allow each first permanent magnet to oscillate at a resonant frequency about an equilibrium position with respect to each second permanent magnet in response to a magnetic or electromagnetic field.

The system can include one or more electromagnetic sources external to the one or more micromechanical resonators 150. The one or more electromagnetic sources can provide external magnetic or electromagnetic fields to excite the respective resonant frequencies of the micromechanical resonators 150. For example, the one or more electromagnetic sources can be the electro-magnetic transducers 122 described with reference to FIG. 1 which emit magnetic or electromagnetic fields 166 to excite the respective resonant frequencies of the micromechanical resonators 150.

The system can include one or more sensors external to the one or more micromechanical resonators 150. The one or more sensors can be configured to determine respective positions of the one or more micromechanical resonators 150 based on sensing the respective resonant frequencies. For example, the one or more electromagnetic transducers 122 can be configured in a receive mode in which the one or more electromagnetic transducers 122 sense disturbances in a magnetic or electromagnetic field due to the oscillations of the first magnetic object 220 within the respective micromechanical resonators 150, as described above with reference to FIGS. 3 and 4.

Since each micromechanical resonator 150 has a different resonant frequency, a Fourier transformation of the signal measured by the one or more electromagnetic sources 122 can reveal a spike at the respective resonant frequencies for each micromechanical resonator 150. A computer can use triangulation to determine the coordinates of each micromechanical resonator 150 by processing the portion of the signal associated with each resonant frequency spike of all electromagnetic sources 122. The frequency can be used to separate mechanical resonators and the intensity of the signal sent back from the mechanical resonator to the different coils can be used to determine the position of the mechanical resonators.

The system can include a medical device with the one or more micromechanical resonators 150 disposed in the medical device. In some examples, the micromechanical resonators 150 are embedded in the medical device. For example, the computer is configured to determine a position of the medical device based on sensing the respective resonant frequencies. In some examples, the medical device is a catheter (e.g., catheter 118) and the one or more micromechanical resonators 150 are inside the catheter 118 (e.g., embedded in a sidewall 117 of the catheter 118). In such examples, the one or more micromechanical resonators 150 are allowed to move relative to one another as the catheter 118 bends through a tortuous anatomy of a patient.

A number of systems and methods have been described. Nevertheless, it will be understood that various modifications may be made without departing from the spirit and scope of this disclosure.

While the micromechanical resonators have been described as including two magnetic objects, some micromechanical resonators include three magnetic objects. For example, consider the micromechanical resonator 900 described with reference to FIG. 21 with an additional, third magnetic object disposed axially between the first and second magnetic objects 220, 902. The third magnetic object can be fixed to the housing. In such a case, the magnetic axis of the second magnetic object 902 can be oriented in the same direction as the magnetic axis of the first magnetic object 220 with a magnetic axis of the third magnetic object oriented in the opposite direction as the first and second magnetic objects 220, 902.

Some micromechanical resonators can include movable permanent magnets as the first magnetic object combined with coils as the second magnetic object. Some micromechanical resonators can include movable coils as the first magnetic object combined with permanent magnets as the second magnetic object.

While the micromechanical resonators have been described with reference to a medical environment of FIG. 1, other applications are possible. For example, using micromechanical resonators to track the 3D position of small animals (e.g., mice, rodents, hamsters, etc.) in a controlled cage (e.g., by attaching the micromechanical resonators in or on the small animals). For example, using micromechanical resonators to track a 3D position of a gesture of a person for consumer gaming or human-machine interface navigation (e.g., by attaching the micromechanical resonators in or on a hand of the person, by attaching the micromechanical resonators in or on a controller to be held by the person, etc.). Other non-exhaustive applications include industrial automation/manufacturing, robotics, skill development/training, military, industrial sensing, etc.

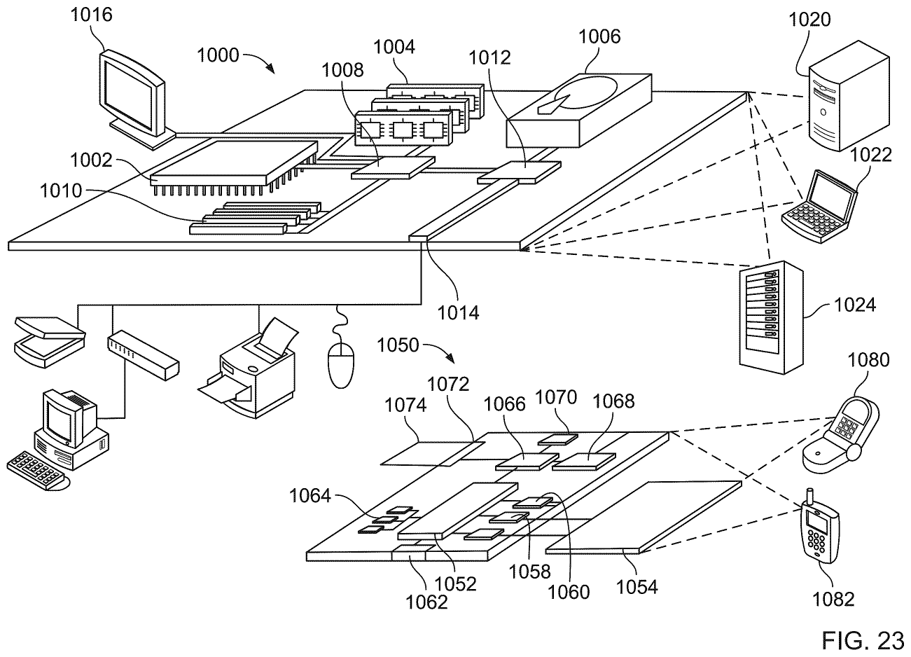

FIG. 23 shows an example computer device 1000 and example mobile computer device 1050 which can be used to implement the techniques described herein. For example, the computer devices 1000, 1050 can execute software to determine the position and/or orientation of the micromechanical resonators described herein based on signals received from the one or more electromagnetic sources/receivers 122. As described above, the position and/or orientation of the micromechanical resonators can be used to infer the position and/or orientation of the medical instrument to which the micromechanical resonators are attached (e.g., the catheter 118). The computer devices 1000, 1050 can then present this information on a display for the medical professional to assist in guiding the medical instrument to the desired location within the anatomy of a patient. In some implementations, the computer system 108 and/or the electromagnetic sources/receivers (e.g., electromagnetic transducer 122) include one or more components of the example computer device 900 and example mobile computer device 950.

Computing device 1000 is intended to represent various forms of digital computers, including, e.g., laptops, desktops, workstations, personal digital assistants, servers, blade servers, mainframes, and other appropriate computers. Computing device 1054 is intended to represent various forms of mobile devices, including, e.g., personal digital assistants, tablet computing devices, cellular telephones, smartphones, and other similar computing devices. The components shown here, their connections and relationships, and their functions, are meant to be examples only, and are not meant to limit implementations of the techniques described and/or claimed in this document.

Computing device 1000 includes processor 1002, memory 1004, storage device 1006, high-speed interface 1008 connecting to memory 1004 and high-speed expansion ports 1010, and low speed interface 1012 connecting to low speed bus 1014 and storage device 1006. Each of components 1002, 1004, 1006, 1008, 1010, and 1012, are interconnected using various busses, and can be mounted on a common motherboard or in other manners as appropriate. Processor 1002 can process instructions for execution within computing device 1000, including instructions stored in memory 1004 or on storage device 1006 to display graphical data for a GUI on an external input/output device, including, e.g., display 1016 coupled to high speed interface 1008. In other implementations, multiple processors and/or multiple busses can be used, as appropriate, along with multiple memories and types of memory. Also, multiple computing devices 1000 can be connected, with each device providing portions of the necessary operations (e.g., as a server bank, a group of blade servers, or a multi-processor system).

Memory 1004 stores data within computing device 1000. In one implementation, memory 1004 is a volatile memory unit or units. In another implementation, memory 1004 is a non-volatile memory unit or units. Memory 1004 also can be another form of computer-readable medium (e.g., a magnetic or optical disk). Memory 1004 may be non-transitory.

Storage device 1006 is capable of providing mass storage for computing device 1000. In one implementation, storage device 1006 can be or contain a computer-readable medium (e.g., a floppy disk device, a hard disk device, an optical disk device, a tape device, a flash memory, or other similar solid state memory device, or an array of devices, such as devices in a storage area network or other configurations.) A computer program product can be tangibly embodied in a data carrier. The computer program product also can contain instructions that, when executed, perform one or more methods (e.g., those described above.) The data carrier is a computer-or machine-readable medium, (e.g., memory 1004, storage device 1006, memory on processor 1002, and the like.)

High-speed controller 1008 manages bandwidth-intensive operations for computing device 1000, while low speed controller 1012 manages lower bandwidth-intensive operations. Such allocation of functions is an example only. In one implementation, high-speed controller 1008 is coupled to memory 1004, display 1016 (e.g., through a graphics processor or accelerator), and to high-speed expansion ports 1010, which can accept various expansion cards (not shown). In the implementation, low-speed controller 1012 is coupled to storage device 1006 and low-speed expansion port 1014. The low-speed expansion port, which can include various communication ports (e.g., USB, Bluetooth®, Ethernet, wireless Ethernet), can be coupled to one or more input/output devices, (e.g., a keyboard, a pointing device, a scanner, or a networking device including a switch or router, e.g., through a network adapter.)

Computing device 1000 can be implemented in a number of different forms, as shown in the FIG. 23. For example, it can be implemented as standard server 1020, or multiple times in a group of such servers. It also can be implemented as part of rack server system 1024. In addition or as an alternative, it can be implemented in a personal computer (e.g., laptop computer 1022.) In some examples, components from computing device 1000 can be combined with other components in a mobile device (not shown), e.g., device 1054. Each of such devices can contain one or more of computing device 1000, 1054, and an entire system can be made up of multiple computing devices 1000, 1054 communicating with each other.

Computing device 1054 includes processor 1052, memory 1064, an input/output device (e.g., display 1054, communication interface 1066, and transceiver 1068) among other components. Device 1054 also can be provided with a storage device, (e.g., a microdrive or other device) to provide additional storage. Each of components 1054, 1052, 1064, 1054, 1066, and 1068, are interconnected using various buses, and several of the components can be mounted on a common motherboard or in other manners as appropriate.

Processor 1052 can execute instructions within computing device 1054, including instructions stored in memory 1064. The processor can be implemented as a chipset of chips that include separate and multiple analog and digital processors. The processor can provide, for example, for coordination of the other components of device 1054, e.g., control of user interfaces, applications run by device 1054, and wireless communication by device 1054.

Processor 1052 can communicate with a user through control interface 1058 and display interface 1056 coupled to display 1054. Display 1054 can be, for example, a TFT LCD (Thin-Film-Transistor Liquid Crystal Display) or an OLED (Organic Light Emitting Diode) display, or other appropriate display technology. Display interface 1056 can comprise appropriate circuitry for driving display 1054 to present graphical and other data to a user. Control interface 1058 can receive commands from a user and convert them for submission to processor 1052. In addition, external interface 1062 can communicate with processor 1042, so as to enable near area communication of device 1054 with other devices. External interface 1062 can provide, for example, for wired communication in some implementations, or for wireless communication in other implementations, and multiple interfaces also can be used.

Memory 1064 stores data within computing device 1054. Memory 1064 can be implemented as one or more of a computer-readable medium or media, a volatile memory unit or units, or a non-volatile memory unit or units. Expansion memory 1074 also can be provided and connected to device 1054 through expansion interface 1072, which can include, for example, a SIMM (Single In Line Memory Module) card interface. Such expansion memory 1074 can provide extra storage space for device 1054, or also can store applications or other data for device 1054. Specifically, expansion memory 1074 can include instructions to carry out or supplement the processes described above, and can include secure data also. Thus, for example, expansion memory 1074 can be provided as a security module for device 1054, and can be programmed with instructions that permit secure use of device 1054. In addition, secure applications can be provided through the SIMM cards, along with additional data, (e.g., placing identifying data on the SIMM card in a non-hackable manner.)

The memory can include, for example, flash memory and/or NVRAM memory, as discussed below. In one implementation, a computer program product is tangibly embodied in a data carrier. The computer program product contains instructions that, when executed, perform one or more methods, e.g., those described above. The data carrier is a computer-or machine-readable medium (e.g., memory 1064, expansion memory 1074, and/or memory on processor 1052), which can be received, for example, over transceiver 1068 or external interface 1062.

Device 1054 can communicate wirelessly through communication interface 1066, which can include digital signal processing circuitry where necessary. Communication interface 1066 can provide for communications under various modes or protocols (e.g., GSM voice calls, SMS, EMS, or MMS messaging, CDMA, TDMA, PDC, WCDMA, CDMA2000, or GPRS, among others.) Such communication can occur, for example, through radio-frequency transceiver 1068. In addition, short-range communication can occur, e.g., using a Bluetooth®, WiFi, or other such transceiver (not shown). In addition, GPS (Global Positioning System) receiver module 1070 can provide additional navigation-and location-related wireless data to device 1054, which can be used as appropriate by applications running on device 1054. Sensors and modules such as cameras, microphones, compasses, accelerators (for orientation sensing), etc. may be included in the device.

Device 1054 also can communicate audibly using audio codec 1060, which can receive spoken data from a user and convert it to usable digital data. Audio codec 1060 can likewise generate audible sound for a user, (e.g., through a speaker in a handset of device 1054.) Such sound can include sound from voice telephone calls, can include recorded sound (e.g., voice messages, music files, and the like) and also can include sound generated by applications operating on device 1054.

Computing device 1054 can be implemented in a number of different forms, as shown in the FIG. 23. For example, it can be implemented as cellular telephone 1080. It also can be implemented as part of smartphone 1082, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor. The programmable processor can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described here can be implemented on a computer having a device for displaying data to the user (e.g., a CRT (cathode ray tube) or LCD (liquid crystal display) monitor), and a keyboard and a pointing device (e.g., a mouse or a trackball) by which the user can provide input to the computer. Other kinds of devices can be used to provide for interaction with a user as well; for example, feedback provided to the user can be a form of sensory feedback (e.g., visual feedback, auditory feedback, or tactile feedback); and input from the user can be received in a form, including acoustic, speech, or tactile input.

The systems and techniques described here can be implemented in a computing system that includes a backend component (e.g., as a data server), or that includes a middleware component (e.g., an application server), or that includes a frontend component (e.g., a client computer having a user interface or a Web browser through which a user can interact with an implementation of the systems and techniques described here), or a combination of such back end, middleware, or frontend components. The components of the system can be interconnected by a form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Accordingly, other implementations are within the scope of the following claims.

1. A device comprising an elongated housing comprising a groove at a longitudinal center of a first end the elongated housing;
a filament having a first end disposed within the groove and being attached to the elongated housing;
a first permanent magnet disposed in the elongated housing and being connected to a second end of filament, the first permanent magnet having a cylindrical portion; and
a second permanent magnet disposed in the elongated housing and being attached to the elongated housing at a second end of the elongated housing, the second permanent magnet having a cylindrical portion and being axially spaced apart from the first permanent magnet,
wherein the filament is configured to allow the first permanent magnet to oscillate about an equilibrium position with respect to the second permanent magnet in response to a magnetic or electromagnetic field.

2. The device of claim 1, wherein a lateral side of the groove is open to an enlarged opening of the elongated housing.

3. The device of claim 2, wherein the enlarged opening is configured to allow axial insertion of the filament through the first end of the elongated housing before the filament is laterally moved into the groove.

4. The device of claim 2, wherein the groove is recessed into a sidewall of the elongated housing defining the enlarged opening.

5. The device of claim 2, wherein the enlarged opening is disposed within a reservoir at the first end of the elongated housing.

6. The device of claim 1, wherein the elongated housing comprises one or more resilient arms disposed at the second end of the elongated housing, the one or more resilient arms configured to apply a radially inward force to the second permanent magnet for holding the second permanent magnet in a fixed location within the elongated housing.

7. The device of claim 6, wherein the one or more resilient arms extend in a longitudinal direction and surround the second permanent magnet.

8. The device of claim 7, wherein an axial end of the one or more resilient arms has a tapered internal diameter to allow outward radial deflection of the one or more resilient arms when the second permanent magnet is inserted into the elongated housing, and the outward radial deflection of the one or more resilient arms causes the radially inward force on the second permanent magnet to be applied.

9. The device of claim 6, wherein the device is configured such that prior to the second permanent magnet being adhered to the one or more resilient arms, the one or more resilient arms (i) allow the second permanent magnet to move axially relative to the elongated housing when an axial force exerted on the second permanent magnet exceeds a threshold force and (ii) hold the second permanent magnet axially relative to the elongated housing when the axial force exerted on the second permanent magnet is below the threshold force.

10. The device of claim 9, wherein the device is configured such that prior to the second permanent magnet being adhered to the one or more resilient arms, an axial movement of the second permanent magnet relative to the elongated housing changes a resonant frequency in which the first permanent magnet oscillates about a longitudinal axis of the elongated housing relative to the second permanent magnet.

11. The device of claim 1, comprising a first part attached to (i) a first end of the first permanent magnet and (ii) the second end of the filament.

12. The device of claim 11, wherein the first part is configured to concentrically align the first permanent magnet to the elongated housing.

13. The device of claim 11, wherein the first part comprises one or more openings extending through the first part, and the filament extends through the one or more openings of the first part.

14. The device of claim 13, wherein at least one opening of the one or more openings has a narrow portion located at a longitudinal axis of the first part and a wide portion that is open to the narrow portion, the narrow portion being open at an outer perimeter of the first part for allowing lateral insertion of the filament into the first part.

15. The device of claim 14, wherein the at least one opening is tapered along the longitudinal axis of the first part.

16. The device of claim 13, wherein the one or more openings of the first part are configured to allow the filament to be inserted into the one or more openings in a lateral direction that is perpendicular to a longitudinal axis of the first part.

17. The device of claim 13, wherein the one or more openings of the first part comprise two or more openings that are spaced apart from one another around a longitudinal axis of the first part.

18. The device of claim 17, wherein the filament extends through the two or more openings and is at least partially wound around the first part.

19. The device of claim 11, wherein the first part comprises a first axially-extending protrusion, and the filament is attached to the first axially-extending protrusion.

20. The device of claim 11, wherein the first part comprises a plurality of distance elements integrally formed with the first part, the plurality of distance elements being configured to allow the first part to be substantially parallel to the first permanent magnet when the first part is in contact with the first permanent magnet.

21. The device of claim 1, wherein a magnetic axis of the first permanent magnet is oriented in a first direction that is perpendicular to a longitudinal axis of the elongated housing, and a magnetic axis of the second permanent magnet is oriented in a second direction that is opposite to the first direction.

22. The device of claim 21, wherein the first direction is perpendicular to a longitudinal axis of the cylindrical portion of the first permanent magnet, and the second direction is perpendicular to a longitudinal axis of the cylindrical portion of the second permanent magnet.

23. The device of claim 1, wherein the filament comprises a Kevlar® fiber, an ultra-high density polyethylene fiber, a carbon fiber, or an aramid fiber, and a torsional stiffness of the filament is less than 1e-7 Nm/rad, and an axial stiffness of the filament is greater than 1 GPa.

24. The device of claim 1, wherein at least one of the first permanent magnet or the second permanent magnet has a tapered cylindrical portion.

25. The device of claim 1, wherein the first permanent magnet has a first tapered cylindrical portion, the second permanent magnet has a second tapered cylindrical portion, and the first and second tapered cylindrical portions directly face one another.

26-65. (canceled)
